(19) United States
(12) Patent Application Publication
(10) Pub. No.: US 2025/0129334 A1
(43) Pub. Date: Apr. 24, 2025

(54) LINEAGE SPECIFICATION DURING STEM CELL TRANSITION

(71) Applicant: Amniotics AB, Lund (SE)

(72) Inventor: Niels-Bjarne Woods, Furulund (SE)

(21) Appl. No.: 18/696,080

(22) PCT Filed: Oct. 3, 2022
(86) PCT No.: PCT/EP2022/077490

§ 371 Date: Mar. 27, 2024

Related U.S. Application Data
(60) Provisional application No. 63/307,937, filed on Feb. 8, 2022.
(60) Provisional application No. 63/365,223, filed on May 24, 2022.

(30) Foreign Application Priority Data
Oct. 1, 2021 (SE) 2130271-6

(51) Int. Cl. C12N 5/078

(52) U.S. Cl. CPC C12N 5/0634; C12N 2501/999; C12N 2506/03; C12N 2506/11

(57) ABSTRACT

A method of generating a hematopoietic cell, including providing a source cell and treating the source cell with a metabolic regulator that directs the source cell to preferentially use glycolysis or oxidative phosphorylation and/or cholesterol biosynthesis, wherein the source cell is differentiated into a GPA⁺ erythroid cell or a CD45⁺ non-erythroid cell. In some examples, the source cell is selected from the group consisting of a hemogenic endothelial (HE) cell, a iPS cell such as a differentiating iPS cell, a cell directly reprogrammed to a known pre-cursor of a hematopoietic cell, a cell directly reprogrammed to a hematopoietic cell or precursor of a hematopoietic cell, a reprogrammed cell that is subsequently further reprogrammed to a hematopoietic cell or precursor of a hematopoietic cell, an adult hematopoietic cell derived from bone marrow or mobilized peripheral blood and a neonatal hematopoietic cell derived from cord blood or prenatal tissue (e.g. placenta). Some examples involve metabolically regulating a lipid biosynthesis pathway with an inhibitor of the lipid biosynthesis pathway or metabolically regulating a histone acetylation pathway with an inhibitor of the histone acetylation pathway.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD OF THE INVENTION

Methods of generating hematopoietic cells from differentiating source cells selected from the group consisting of induced pluripotent stem cells (iPS), cells directly reprogrammed to pre-cursors of hematopoietic cells, cells directly reprogrammed to hematopoietic cells, and adult or neonatal hematopoietic cells derived from bone marrow, cord blood, prenatal tissue (e.g. placenta), or mobilized peripheral blood.

### BACKGROUND

In the developing embryo, primitive hematopoiesis gives rise to erythrocytes, megakaryocytes and macrophages in the blood islands of the yolk sac (YS) (Palis, J., Robertson, S., Kennedy, M., Wall, C. & Keller, G. Development of erythroid and myeloid progenitors in the yolk sac and embryo proper of the mouse. Development 126, 5073-5084 (1999)). Next, a definitive wave of hematopoiesis produces more mature erythro-myeloid and lymphoid (Yoder, M. C. et al. Characterization of Definitive Lymphohematopoietic Stem Cells in the Day 9 Murine Yolk Sac. Immunity 7, 335-344 (1997); and Böiers, C. et al. Lymphomyeloid Contribution of an Immune-Restricted Progenitor Emerging Prior to Definitive Hematopoietic Stem Cells. Cell Stem Cell 13, 535-548 (2013)) progenitors. Around Carnegie stage (CS) (12-13), hematopoietic stem cells (HSCs) emerge in the aorta-gonad-mesonephros (AGM) region through a second definitive hematopoietic wave (Medvinsky, A. & Dzierzak, E. Definitive Hematopoiesis Is Autonomously Initiated by the AGM Region. Cell 86, 897-906 (1996); and Ivanovs, A. et al. Highly potent human hematopoietic stem cells first emerge in the intraembryonic aorta-gonad-mesonephros region. J Exp Med 208, 2417-2427 (2011)). Primitive erythrocytes, erythro-myeloid progenitors (EMPs) and HSCs derive from a hemogenic endothelial (HE) cell (Lancrin, C. et al. The haemangioblast generates haematopoietic cells through a haemogenic endothelium stage. Nature 457, 892-895 (2009); Frame, J. M., Fegan, K. H., Conway, S. J., McGrath, K. E. & Palis, J. Definitive Hematopoiesis in the Yolk Sac Emerges from Wnt-Responsive Hemogenic Endothelium Independently of Circulation and Arterial Identity. STEM CELLS 34, 431-444 (2016); and Stefanska, M. et al. Primitive erythrocytes are generated from hemogenic endothelial cells. Sci Rep 7, 1-10 (2017)) by a process known as endothelial to hematopoietic transition (EHT) (Boisset, J.-C. et al. In vivo imaging of haematopoietic cells emerging from the mouse aortic endothelium. Nature 464, 116-120 (2010); and Kissa, K. & Herbomel, P. Blood stem cells emerge from aortic endothelium by a novel type of cell transition. Nature 464, 112-115 (2010)). Studies on hematopoietic emergence during embryonic development have not only described EHT in spatial and temporal contexts in several animal models (Boisset, J.-C. et al. In vivo imaging of haematopoietic cells emerging from the mouse aortic endothelium. Nature 464, 116-120 (2010); and Kissa, K. & Herbomel, P. Blood stem cells emerge from aortic endothelium by a novel type of cell transition. Nature 464, 112-115 (2010)) but also led to a deep understanding of the growth and transcription factors regulating this process (Chen, M. J., Yokomizo, T., Zeigler, B. M., Dzierzak, E. & Speck, N. A. Runxl is required for the endothelial to haematopoietic cell transition but not thereafter. Nature 457, 887-891 (2009); Zhou, F. et al. Tracing haematopoietic stem cell formation at single-cell resolution. Nature 533, 487-492 (2016); and Swiers, G. et al. Early dynamic fate changes in haemogenic endothelium characterized at the single-cell level. Nat Commun 4, 2924 (2013)). Some studies of the transcriptional landscape in mouse models have hinted towards an increase in metabolic processes during HSC emergence (Zhou, F. et al. Tracing haematopoietic stem cell formation at single-cell resolution. Nature 533, 487-492 (2016); Gao, P. et al. Transcriptional regulatory network controlling the ontogeny of hematopoietic stem cells. Genes Dev. (2020) doi:10.1101/gad.338202.120; and Oatley, M. et al. Single-cell transcriptomics identifies CD44 as a marker and regulator of endothelial to haematopoietic transition. Nat Commun 11, 1-18 (2020)).

Growing evidence points to the fact that metabolic pathways can control cell fate (Oburoglu, L. et al. Glucose and Glutamine Metabolism Regulate Human Hematopoietic Stem Cell Lineage Specification. Cell Stem Cell 15, 169-184 (2014); Moussaieff, A. et al. Glycolysis-Mediated Changes in Acetyl-CoA and Histone Acetylation Control the Early Differentiation of Embryonic Stem Cells. Cell Metabolism 21, 392-402 (2015) and Folmes, C. D. L. et al. Somatic Oxidative Bioenergetics Transitions into Pluripotency-Dependent Glycolysis to Facilitate Nuclear Reprogramming. Cell Metabolism 14, 264-271 (2011)). Specifically, the fate of bone marrow (BM) HSCs is regulated by several metabolic pathways. The hypoxic niche of the BM pushes HSCs to activate a minimal energy-providing pathway, anaerobic glycolysis, and ensures their quiescent state (Takubo, K. et al. Regulation of Glycolysis by Pdk Functions as a Metabolic Checkpoint for Cell Cycle Quiescence in Hematopoietic Stem Cells. Cell Stem Cell 12, 49-61 (2013)). HSC self-renewal and maintenance rely on fatty acid oxidation (Ito, K. et al. A PML-PPAR-S pathway for fatty acid oxidation regulates hematopoietic stem cell maintenance. Nat Med 18, 1350-1358 (2012)) and differentiating HSCs switch to oxidative phosphorylation (OXPHOS) to meet their energetic requirements (Yu, W.-M. et al. Metabolic Regulation by the Mitochondrial Phosphatase PTPMT1 Is Required for Hematopoietic Stem Cell Differentiation. Cell Stem Cell 12, 62-74 (2013); and Simsek, T. et al. The Distinct Metabolic Profile of Hematopoietic Stem Cells Reflects Their Location in a Hypoxic Niche. Cell Stem Cell 7, 380-390 (2010)).

The EHT process has been modelled extensively in vitro using pluripotent stem cells (PSCs) and the HE intermediate which arises in this context can give rise to both primitive and definitive hematopoietic cells (Garcia-Alegria, E. et al. Early Human Hemogenic Endothelium Generates Primitive and Definitive Hematopoiesis In Vitro. Stem Cell Reports 11, 1061-1074 (2018)). Several studies have focused on obtaining HE with definitive potential in vitro (Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012); Sugimura, R. et al. Haematopoietic stem and progenitor cells from human pluripotent stem cells. Nature 545, 432-438 (2017); Ng, E. S. et al. Differentiation of human embryonic stem cells to HOXA+ hemogenic vasculature that resembles the aorta-gonad-mesonephros. Nature Biotechnology 34, 1168-1179 (2016) and Sturgeon, C. M., Ditadi, A., Awong, G., Kennedy, M. & Keller, G. Wnt signaling controls the specification of definitive and primitive hematopoiesis from human pluripotent stem cells. Nat Biotech 32, 554-561 (2014)), by modulating various signaling pathways, in an effort to gain further insight into definitive hematopoietic cell development to ultimately produce functional and transplantable HSCs for therapeutic use. In this study, we set out to uncover whether metabolic modulations could prompt HE cells to preferentially adopt a definitive hematopoietic fate.

As EHT implicates tight-junction dissolution, gain of stem cell-like properties and leads to extensive transcriptional and phenotypic changes in the transitioning cell (Zhou, F. et al. Tracing haematopoietic stem cell formation at single-cell resolution. Nature 533, 487-492 (2016); Swiers, G. et al. Early dynamic fate changes in haemogenic endothelium characterized at the single-cell level. Nat Commun 4, 2924 (2013); and Guibentif, C. et al. Single-Cell Analysis Identifies Distinct Stages of Human Endothelial to-Hematopoietic Transition. Cell Reports 19, 10-19 (2017)), we hypothesized that metabolism contributes to regulating these processes. Previously, in animal models, the emergence of HSCs was shown to be regulated by adenosine signaling and the PKA-CREB pathway (Jing, L. et al. Adenosine signaling promotes hematopoietic stem and progenitor cell emergence. J Exp Med 212, 649-663 (2015); and Kim, P. G. et al. Flow-induced protein kinase A-1 CREB pathway acts via BMP signaling to promote HSC emergence. J Exp Med 212, 633-648 (2015)), which are tightly controlled by ATP levels and availability; suggesting a change in energy demand during EHT. Moreover, glucose metabolism was shown to induce HSC emergence in zebrafish (Harris, J. M. et al. Glucose metabolism impacts the spatiotemporal onset and magnitude of HSC induction in vivo. Blood 121, 2483-2493 (2013)). However, use of metabolites and metabolic pathways to drive the emergence of hematopoietic cells has not been evaluated at length during development and such mechanisms may be useful for the production of particular stem cell lineages. Therefore, there is a need for methods and compositions that utilize metabolites and/or metabolic pathways to drive stem cell differentiation.

### SUMMARY OF THE INVENTION

Some embodiments relate to a method of generating a hematopoietic cell, including:
providing a source cell; and
treating the source cell with a metabolic regulator that directs the source cell to preferentially use glycolysis or oxidative phosphorylation; and
thereby obtaining a GPA+ erythroid cell or a CD45+ non-erythroid cell (which may also be referred to herein as a CD45+ hematopoietic cell).

It is routine to measure glycolysis and oxidative phosphorylation, for example by using a commercially available kit. Accordingly, the preferential use of glycolysis could be assessed, for example by using a glycolysis assay kit, and comparing a source cell treated with a metabolic regulator with an appropriate control, such as a source cell that has not been treated with a metabolic regulator. Identifying a higher rate of glycolysis may indicate preferential glycolysis for the source cell treated with a metabolic regulator. On the other hand, the preferential use of oxidative phosphorylation could be assessed, and a comparison made between a source cell treated with a metabolic regulator and an appropriate control, such as a source cell that has not been treated with a metabolic regulator. Identifying a higher rate of oxidative phosphorylation may indicate preferential oxidative phosphorylation for the source cell treated with a metabolic regulator. Alternatively, both glycolysis and oxidative phosphorylation could be assessed.

In some examples, the source cell is selected from the group consisting of a hemogenic endothelial (HE) cell, an iPS cell (e.g. a differentiating iPS cell), a cell directly reprogrammed to a known pre-cursor of a hematopoietic cell, a cell directly reprogrammed to a hematopoietic cell or precursor of a hematopoietic cell, a reprogrammed cell that is subsequently further reprogrammed to a hematopoietic cell or precursor of a hematopoietic cell, an adult hematopoietic cell derived from bone marrow or mobilized peripheral blood and a neonatal hematopoietic cell derived from cord blood or prenatal tissue (e.g. placenta).

In some examples, the metabolic regulator is a molecule selected from the group consisting of a drug, a protein, an RNA based system that regulates metabolic processes and any combination thereof.

In some examples, the metabolic regulator is selected from the group consisting of a viral vector, an RNA-based system, a CRISPR/CAS-based system that regulates metabolic processes and any combination thereof.

In some examples, the metabolic regulator is also combinations of 1 or more molecules selected from the group consisting of drugs, proteins, viral vectors, RNA-based systems, CRISPR/CAS-based systems that specifically regulate metabolic processes and any combination thereof.

In some examples, the source cell is directed to glycolysis by blocking pyruvate metabolism to generate GPA+ erythroid cells. For example, by blocking pyruvate entry to the mitochondria and limiting TCA cycle activity and/or OXPHOS, GLY+ erythroid cells can be generated. Alternatively, driving TCA cycle and/or OXPHOS via feeding the source cell with pyruvate (or similar) yields CD45+ non-erythroid cells. GPA+ and GLY+ are abbreviations for glycophorin A and are used herein interchangeably.

In some examples, pyruvate is blocked from entering mitochondria, thereby inhibiting tricarboxylic acid (TCA) cycle activity. The term “inhibiting”, as used herein, is used interchangeably with “reducing”, “silencing”, “downregulating”, “suppressing” and other similar terms, and includes any level of inhibition. In certain embodiments, a level of inhibition, e.g. for a metabolic regulator described herein, can be assessed in cell culture conditions. The inhibition of TCA cycle activity may be assessed, for example, by measuring the activity of one or more enzyme involved with the TCA cycle. For example, a metabolite derived from an enzyme involved with the TCA cycle may be quantified prior to exposure to the enzyme in the TCA cycle. A decrease in that metabolite would indicate that the activity of the TCA cycle has been inhibited. Alternatively, or additionally, the metabolite derived from an enzyme involved with the TCA cycle may be quantified after exposure to the enzyme in the TCA cycle. An increase in that metabolite would indicate that the activity of the TCA cycle has been enhanced (or no change in the quantified amount may indicate that the enzyme is not inhibited).

In some examples, the metabolic regulator blocks:
(a) mitochondrial pyruvate carrier (MPC); or
(b) pyruvate dehydrogenase complex (PDH).

In some examples, the metabolic regulator that blocks MPC is UK5099, which inhibits MPC. Inhibition of MPC may be assessed by checking the detectable levels of a metabolite (e.g. pyruvate) that is transported by MPC before and after treatment with a metabolic regulator, or by comparing detectable levels of a metabolite (e.g. pyruvate) that is transported by MPC between a source cell that has been treated with a metabolic regulator and a control source cell that has not been treated with a metabolic regulator. For example, the detection of the metabolite may be based on its quantification in the cytoplasm and/or the mitochondria of a cell (wherein a decrease from the cytoplasm indicates increased transport into the mitochondria).

In some examples, the metabolic regulator that blocks PDH is 1-AA, which inhibits PDH. PDH is a complex of three enzymes that converts pyruvate into acetyl-CoA. Therefore, in some cases, inhibition of PDH may be assessed by checking the detectable levels of pyruvate and/or acetyl-CoA before and after treatment with a metabolic regulator that blocks PDH (e.g. 1-AA), or by comparing detectable levels of pyruvate and/or acetyl-CoA between a source cell that has been treated with a metabolic regulator that blocks PDH (e.g. 1-AA) and a control source cell that has not been treated with the metabolic regulator.

In some examples, the expression of MPC subunits (MPC1 and/or MPC2) is/are downregulated using shRNAs. The downregulation of the MPC subunits may be assessed in comparison with a relevant control (i.e. a control source cell that has not been treated with a metabolic regulator). Downregulation can be quantified as a percentage of the level of MPC subunits detectable after treatment with a metabolic regulator in comparison with the level of MPC subunits prior to said treatment.

In some examples, the source cell is directed to promote OXPHOS or tricarboxylic acid (TCA) cycle activity to generate CD45+ hematopoietic cells. By “promote”, we include the meaning that a higher level of OXPHOS or TCA cycle activity is occurring following treatment with the metabolic regulator, i.e. the metabolic regulator causes the source cell to undergo a higher level of OXPHOS, or a higher level of activity in the TCA cycle.

In some examples, the metabolic regulator that promotes tricarboxylic acid (TCA) cycle activity is selected from the group consisting of dimethyl α-ketoglutarate (DMK), alpha-ketoglutarate, a related molecule and any combination thereof.

In some examples, the source cell is directed to use pyruvate via oxidative phosphorylation (OXPHOS) and the source cell is differentiated into definitive CD45+ non-erythroid cells.

In some examples, the metabolic regulator promotes pyruvate use by the TCA cycle. The promotion of pyruvate use can be assessed by measuring a starting level of pyruvate in a source cell, and comparing that measurement to the level of pyruvate following treatment with a metabolic regulator. If the level of pyruvate decreases, then the metabolic regulator promotes use of pyruvate by the TCA cycle. Alternatively, or additionally, the metabolites of pyruvate following the TCA cycle may be measured, before and after treatment with a metabolic regulator, wherein an increase in at least one metabolite of pyruvate indicates that the metabolic regulator promotes use of pyruvate by the TCA cycle. These approaches could be coupled with known assays for assessing TCA cycle activity.

In some examples, DCA or a related molecule or an shRNA is used to inhibit pyruvate dehydrogenase kinases (PDKs) and thereby promote pyruvate use by OXPHOS.

In some examples, the method includes increasing pyruvate flux into mitochondria, which amplifies acetyl-CoA production and in turn promotes cholesterol metabolism and favors definitive hematopoietic output.

In some examples, the CD45+ non-erythroid cells are lymphoid cells. In some examples, the lymphoid cells are innate lymphoid cells (ILCs), optionally selected from the group consisting of ILC1s, ILC2s, ILC3s and combinations thereof.

In some examples, the lymphoid cells are T and/or B cells.

In some examples, the lymphoid cells are NK cells.

In some examples, the lymphoid cells are NKT cells.

In some examples, the lymphoid cells are lymphoid progenitors selected from the group consisting of common lymphoid progenitors (CLPs), pro-B cells, pre-B cells, thymocyte progenitors and precursors, NK cell progenitors and precursors and any combination thereof.

Some examples relate to a method of generating hematopoietic cells, including metabolically regulating a lipid biosynthesis pathway with an inhibitor of the lipid biosynthesis pathway, and thereby obtaining hematopoietic cells (such as a GLY+ erythroid cell or a CD45+ non-erythroid cell).

In some examples, the inhibitor of the lipid biosynthesis pathway is CP-640186.

Some examples relate to a method of generating hematopoietic cells including metabolically regulating a histone acetylation pathway with an inhibitor of the histone acetylation pathway, and thereby obtaining hematopoietic cells (such as a GLY+ erythroid cell or a CD45+ non-erythroid cell).

In some examples, the inhibitor of the histone acetylation pathway is C646.

In some examples, the metabolic regulator includes an inhibitor or activator targeting glutaminolysis. Glutaminolysis is a series of reactions where the glutamine is lysed to glutamate, aspartate, CO₂, pyruvate, lactate, alanine and citrate. Therefore, a metabolic regulator that is an inhibitor targeting glutaminolysis may be assessed by measuring the levels of any of the metabolites derived from the lysis of glutamine, as a comparison prior to and after treatment with a metabolic regulator (or by comparing with an untreated source cell control), wherein a decrease in the level of one or more metabolite of glutamine in the treated cell indicates that the metabolic regulator includes an inhibitor targeting glutaminolysis. On the other hand, a metabolic regulator that is an activator targeting glutaminolysis may be assessed by measuring the levels of any of the metabolites derived from the lysis of glutamine, as a comparison prior to and after treatment with a metabolic regulator (or by comparing with an untreated source cell control), wherein an increase in the level of one or more metabolite of glutamine in the treated cell indicates that the metabolic regulator includes an inhibitor targeting glutaminolysis. Alternatively, or additionally, the inhibition or activation may be assessed based on a starting concentration of detectable glutamine (i.e. a baseline or background level) prior to treatment with a metabolic regulator, and an ending concentration of detectable glutamine after treatment with a metabolic regulator, wherein a lower concentration of glutamine following treatment indicates an activator, and a higher concentration of glutamine before treatment (or no change in concentration following treatment) indicates an inhibitor.

In some examples, the method further includes metabolically regulating alpha-ketoglutarate-dependent histone and DNA methylation with an inhibitor or activator targeting alpha-ketoglutarate-dependent histone and DNA methylation. Assays for measuring DNA methylation states are known to the skilled person. Therefore, it will be readily appreciated that an inhibitor targeting alpha-ketoglutarate-dependent histone and DNA methylation results in a lower level of DNA methylation (compared with a relevant control, such as an untreated control condition); and an activator targeting alpha-ketoglutarate-dependent histone and DNA methylation results in a higher level of DNA methylation (compared with a relevant control, such as an untreated control condition).

In some examples, the method further includes metabolically regulating a glutamine-dependent pathway with an inhibitor or activator targeting the glutamine-dependent pathway. A glutamine-dependent pathway may be a pathway in which glutamine is a metabolite processed by an enzyme within the pathway. Therefore, an inhibitor or activator targeting a glutamine-dependent pathway may be assessed based on a measurement of glutamine levels before and after treatment with a metabolic regulator, wherein no change or an increase in glutamine levels following treatment indicates an inhibitor, and a decrease in glutamine levels following treatment indicates an activator.

In some examples, the glutamine-dependent pathway is selected from the group consisting of nucleotide (purine and pyrimidine) biosynthesis, glutathione synthesis and non-essential amino acid synthesis and any combination thereof.

Some examples relate to a method of generating a hematopoietic cell, including:
providing a source cell; and
treating the source cell with cholesterol or a derivative of cholesterol, and
thereby obtaining a CD45+ hematopoietic cell.

Some examples relate to a method of generating a hematopoietic cell, including:
providing a source cell;
wherein the source cell is treated with an inhibitor or activator targeting the mevalonate pathway, and
thereby obtaining a GLY+ erythroid cell or a CD45+ hematopoietic cell, respectively.

Some examples relate to a method of generating a hematopoietic cell including:
providing a source cell, and
metabolically regulating the source cell with fatty acids or a derivative of fatty acids, and
thereby obtaining a hematopoietic cell (such as a GLY+ erythroid cell or a CD45+ non-erythroid cell).

Some examples relate to a method of generating a hematopoietic cell including:
providing a source cell; and
metabolically regulating the source cell with a lipid or a derivative of a lipid, and
thereby obtaining a hematopoietic cell (such as a GLY+ erythroid cell or a CD45+ non-erythroid cell).

Some examples relate to a method of generating a hematopoietic cell, including:
providing a source cell; and
treating the source cell with an inhibitor or activator that targets a lipid biosynthesis pathway, and
thereby obtaining a GPA+ erythroid cell or a CD45+ non-erythroid cell.

Some examples relate to a method of providing hematopoietic cells to a subject with a malignancy or hematological disorder including:
(a) obtaining hematopoietic cells according to the methods described herein; and
(b) transplanting the hematopoietic cells into the subject.

In some examples, the hematopoietic cells are modified to express chimeric antigen receptors (CAR). In some examples, the hematopoietic cells modified to express CAR are “T cells redirected for antigen-unrestricted cytokine-initiated killing”, i.e. TRUCKs (also referred to as “4^{th} generation” CAR T cells).

In some examples, the malignancy is selected from the group consisting of leukemia (acute lymphocytic (ALL), chronic lymphocytic (CLL), acute myeloid (AML), chronic myeloid (CML)), myeloma, and lymphoma (Hodgkin's and non-Hodgkin's (NHL), Glioblastoma, glioma, pancreatic malignancies, any other malignancy where a hematopoietic cell transplant could be used) and any combination thereof.

In some examples, the source cell is treated with a molecule that increases HMG-CoA reductase to increase cholesterol biosynthesis, thereby increasing output of NK cells. An increase in HMG-CoA reductase (i.e. the activity thereof) can be assessed based on a measurement of cholesterol. HMG-CoA reductase can be the rate-limiting step in cholesterol biosynthesis in certain circumstances, and so a higher level of cholesterol (for example, following treatment with a metabolic regulator) may be indicative of an increase in HMG-CoA reductase activity. Such activity has been shown to increase the output of NK cells, i.e. the percentage of NK cells detectable in a population of source cells is higher than an untreated control, for example as assessed by flow cytometry.

In some examples, the molecule that increases HMG-CoA reductase is thyroid hormone.

In some examples, the thyroid hormone is added to the source cell during the HE stage to increase NK cell output.

Some examples relate to a population of hematopoietic cells obtained from a source cell that has been treated with a metabolic regulator that directs the source cell to preferentially use glycolysis or oxidative phosphorylation, wherein the source cell is differentiated into a GPA+ erythroid cell or a CD45+ non-erythroid cell.

Some examples relate to the population of hematopoietic cells generated by the any of methods described herein.

Some examples relate to a population of hematopoietic cells for use in treating a subject with a malignancy or hematological disorder. In some examples of the use, the hematopoietic cells are modified to express chimeric antigen receptors (CAR). In some examples, the malignancy is selected from the group consisting of leukemia (acute lymphocytic (ALL), chronic lymphocytic (CLL), acute myeloid (AML), chronic myeloid (CML)), myeloma, and lymphoma (Hodgkin's and non-Hodgkin's (NHL), Glioblastoma, glioma, pancreatic malignancies, any other malignancy where a hematopoietic cell transplant could be used) and any combination thereof. In some examples, the source cell is treated with a molecule that increases HMG-CoA reductase to increase cholesterol biosynthesis, thereby increasing output of NK cells. In some examples, the molecule that increases HMG-CoA reductase is thyroid hormone, optionally wherein the thyroid hormone is added to the source cell during the HE stage to increase NK cell output.

### BRIEF DESCRIPTION OF THE DRAWINGS

Other features and advantages will be apparent from the following detailed description, taken in conjunction with the accompanying drawings of which:

FIG. 1. iPSC-derived cells match primary human EHT populations. iPSC-derived HE, EHT and HSC-like cells were sorted, cultured for 1 day and analyzed by scRNAseq. (a) UMAP visualization of scRNAseq data from HE, EHT and HSC-like cells, colored by sorting phenotype. (b) Heatmap showing expression levels of endothelial and hematopoietic genes in HE, EHT and HSC-like populations. (c) UMAP showing AEC/Hem cluster cells from Carnegie stage (CS) 1336 matched against the HE, EHT and HSC-like populations in FIG. 1, a. (d) Heatmap showing expression levels of endothelial and hematopoietic genes in AEC/Hem cluster cells which have mapped to the HE, EHT and HSC-like populations as shown in FIG. 1, c.

FIG. 2. Glycolysis, oxygen consumption and mitochondrial activity increase during EHT. (a) Extracellular acidification rate (ECAR) was measured in HE (n=24), EHT (n=13) and HSC-like (n=8) cells and glycolytic flux was assessed by extracellular flux analysis. Bar graphs show relative levels±s.e.m. of the indicated processes (from 7 (HE, EHT) or 3 (HSC-like) independent experiments, unpaired t-tests). (b) Dot plots showing gene expression levels of glycolytic enzymes detected by scRNAseq and based on percent expressed (size of the dots) and average level of expression (color intensity). (c) FACS-sorted HE cells were subcultured with or without 2-DG (1 mM). Subculture day 3 representative FSC-A/CD43 plots are shown (n=7, see FIG. 8, d for bar graphs). (d) Subculture day 3 representative GPA/CD43 plots and subculture day 6 CD45/CD43 plots are shown (n=6 and n=5, see FIG. 8, e for bar graphs). (e) Subculture day 3 CellTrace Violet (CTV) fluorescence was assessed by flow cytometry (representative of n=4). (f) 2-NBDG uptake was measured by flow cytometry on day 10 for HE, EHT and HSC-like cells and mean MFI levels±s.e.m. are shown (n=4, paired t-tests). (g) Oxygen consumption rate (OCR) was measured in HE and EHT cells (n=7) and oxidative phosphorylation was assessed by extracellular flux analysis. Bar graphs show relative levels±s.e.m. of the indicated processes (from 3 independent experiments; unpaired t-tests). (h) TMRE fluorescence, with or without 100 μM FCCP treatment, was measured by flow cytometry on day 10 for HE, EHT and HSC-like cells and MFI-MFI FMO levels±s.e.m. relative to HE are shown (n=5, paired t-tests). (i) Basal OCR was measured in day 10 HE (n=6), EHT (n=5) and HSC-1 like (n=4) cells and bar graphs show mean levels±s.e.m. relative to HE (paired t-tests). (j) Live cell imaging of HE and HSC3 like cells stained with TMRE (red) at day 3 of subculture. Representative merged brightfield/TMRE and TMRE images are shown. Scale bars, 100 μm. Bar graphs show means of TMRE staining intensity from all replicate wells across all experiments (HE spindle, n=9; HE round, n=9; HSC-like, n=6, Kruskal-Wallis test with multiple comparisons). (k) Dot plots showing gene expression levels of TCA cycle enzymes detected by scRNAseq and based on percent expressed (size of the dots) and average level of expression (color intensity). ns, not significant, *p<0.05, **p<0.01, ***p<0.001, ****p<0.0001.

FIG. 3. Increasing pyruvate flux into mitochondria in HE cells favors definitive hematopoietic differentiation. (a) Pyruvate is transported into mitochondria via the mitochondrial pyruvate carrier (MPC, inhibitor: UK5099) and converted to acetyl-CoA by pyruvate dehydrogenase complex (PDH, inhibitor: 1-AA). Pyruvate dehydrogenase kinases (PDKs, inhibitor: DCA) negatively regulate PDH activity. (b-e) FACS15 sorted HE cells were subcultured with or without UK5099 (10 μM) or DCA (3 mM). Subculture day 3 representative GPA/CD43 plots (b, d) and subculture day 6 representative CD45/CD43 plots (c, e) are shown (see FIG. 10, a, g, 1 and n for corresponding bar graphs). (f) Ratio of CFU-E to CFU-G, M, GM colonies relative to the control condition obtained from HE cells subcultured with the indicated compounds for 6 days, see FIG. 10, s for percentages (n=5, paired t-test). (g) Fold change in the expression of HBE1 or HBG1-2 transcripts normalized to KLF1 in CFUs obtained from HE cells treated with UK5099 (10 M) or DCA (3 mM) relative to non-treated cells. (h) Percentages of CD45⁺CD56⁺±s.e.m. cells obtained following 35-day co-culture of 3-day subcultured HE cells with OP9-DL1 stroma. During the 3-day subculture, HE cells were treated with the indicated compounds. (n=3, one-way ANOVA test, see FIG. 10, v for plots). (i-k) Pregnant mice were injected with UK5099 or DCA at E9.5 and fetal livers were analyzed at E14.5 by flow cytometry. FL, fetal liver. Levels of T and B cells (i) and LT-HSCs (j) as percentages in fetal liver are shown for control (n=10), UK5099-treated (n=14) and DCA-treated (n=16) conditions (one-way ANOVA test). (k) The ratio of BFU-E to CFU-GM colonies obtained from sorted LT-HSCs are shown (see also data in FIG. 11, f) (one-way ANOVA test). CFU, colony forming unit; BFU, burst forming unit; E, erythroid; M, macrophage; G, granulocyte. (l-p) HE cells co-cultured with OP9-DL1 stroma were treated with DCA for 3 days and transplanted into irradiated NSG mice. Bone marrow (BM) and thymi were harvested on week 12. (l) The percentages±s.e.m. of human CD4⁺CD8⁺ double positive thymocytes in huCD45⁺ cells from the thymus are shown (Control, n=6; DCA, n=7; unpaired t tests). The percentages±s.e.m. of human B cells (m), CLPs (n) from the BM at week 12, as well as myeloid cells from PB at week 8 (o) and myeloid cells from BM at week 12 (p) in huCD45+ cells are shown (Control, n=6; DCA, n=7; unpaired t tests). PB, peripheral blood. ns, not significant, *p<0.05, **p<0.01, ***p<0.001.

FIG. 4. Modulation of pyruvate catabolism affects HE commitment at the single-cell level. (a) Control, UK5099-treated and DCA-treated HE cells were visualized together by UMAP and divided into 7 clusters. (b) Heatmap showing scRNAseq data of endothelial or hematopoietic genes expressed in the 7 clusters. (c) Clusters 6 (559 cells) and 7 (280 cells) were assessed independently and dot plots show expression levels of the indicated genes detected by scRNAseq and based on percent expressed (size of the dots) and average level of expression (color intensity). (d) Dot plots show expression levels of the indicated hematopoietic transcription factors in clusters 6 and 7 for HE ctrl, HE+UK5099 and HE+DCA conditions, detected by scRNAseq and based on percent expressed (size of the dots) and average level of expression (color intensity).

FIG. 5. Pyruvate catabolism affects EHT via distinct mechanisms. (a) FACS-sorted HE cells were subcultured with or without TSA (60 nM). Subculture day 3 CD43 MFI levels and a representative CD43 histogram are shown (n=4, paired t test). (b) Dot plots showing gene expression levels of LSD1, GFI1 and GFI1B detected by scRNAseq and based on percent expressed (size of the dots) and average level of expression (color intensity). (c) FACS-sorted HE cells were subcultured with TCP (300 nM), UK5099 (10 μM) or both and day 3 CD43⁺GPA⁺ cell frequencies±SEM relative to the control are shown (n=4, one-way ANOVA test). (d) HE cells were transduced with shScrambled (shScr) or shLSD1 with or without UK5099 (10 μM) the day 1 after the sort and day 3 CD43+/GPA+ cell frequencies±SEM relative to shScr are presented (n=3, one-way ANOVA test). (e) Acetate can be directly converted to acetyl-CoA by ACSS2 (inhibitor: ACSS2i). Acetyl-CoA is the precursor of acetylation marks, transferred onto histones via histone acetyltransferases (HATs, inhibitor: C646). (f-g) FACS-sorted HE cells were subcultured with ACSS2i (5 μM), DCA (3 mM), or both (n=5, one-way ANOVA test) (f) or with C646 (10 μM), DCA (3 mM), or both (n=3, one-way ANOVA test) (g) and day 6 CD43⁺CD45⁺ cell frequencies±SEM are shown. (h) FACS-sorted HE cells were subcultured with or without DCA (3 mM) for 2 days on coverslips. Staining intensities of H3K9 acetylation and H4K5, 8, 12, 16 acetylation were assessed by confocal microscopy imaging and fold change compared to the control is shown (n=3). (i) Acetyl-coA can be a precursor for lipid biosynthesis via ACC (inhibitor: CP-640186 or CP) or for the mevalonate pathway/cholesterol biosynthesis via HMGCR (inhibitor: Atorvastatin or Ato). (j) FACS-sorted HE cells were subcultured with CP (5 μM), DCA (3 mM) or both and day 6 CD43+CD45+ cell frequencies±SEM relative to the control are shown (n=4, one-way ANOVA test). (k) Cholesterol content in HE cells was measured by confocal microscopy at day 2 of DCA (3 mM) treatment by filipin III staining (n=3, paired t test). (l) Cholesterol content in HE cells was assessed by flow cytometry after intracellular staining with filipin III at day 3 of DCA treatment (n=3, one-way ANOVA test). (m) FACS-sorted HE cells were subcultured with Ato (0.5 μM), DCA (3 mM) or both and day 3 CD43⁺CD45⁺ cell frequencies f SEM relative to the control are shown (n=3 for control/DCA, n=2 with 2 technical replicates for Ato/Ato+DCA, one-way ANOVA test). (n) Dot plots showing gene expression levels of NOTCH1, JAG1 and HES1 detected by scRNAseq and based on percent expressed (size of the dots) and average level of expression (color intensity). (o) Notch1 expression (MFI) in HE-derived CD43⁺CD45⁺ cells at day 3 of subculture with or without DCA (3 mM) was assessed by flow cytometry (n=2, 1-2 technical replicates, unpaired t test). (p) Glycolysis is essential for hematopoietic differentiation of HE cells and inhibiting pyruvate entry into mitochondria (via UK5099 or shMPC1/2) favors a primitive erythroid fate. Increasing pyruvate flux into mitochondria (via DCA or shPDK4) amplifies acetyl-CoA production which fuels cholesterol biosynthesis and promotes definitive hematopoietic differentiation of HE cells. ns, not significant, *p<0.05, **p<0.01, ***p<0.001, ****p<0.0001.

FIG. 6. Generation and characteristics of EHT populations of interest. (a) Schematic of the hematopoietic differentiation system. Following embryoid body setup, BMP4, Activin A, CHIR99021, VEGF and hematopoietic cytokines were added sequentially to induce HE cell formation and EHT. Cells of interest were sorted at day 8 of the protocol. (b) Sorting strategy for obtaining pure HE, EHT and HSC-like cell populations. At day 8 of differentiation, representative plots show the level of CD34⁺ cells following magnetic bead enrichment, separation on the basis of CD43 expression and further gating on CXCR4⁻CD73⁻ and CD90⁺VEcad⁺ for HE and EHT cells; and CD90⁺CD38⁻ for HSC-like cells. (c-d) Pseudotime analysis of EHT populations taking a GO (c) or S/G2M (d) path and corresponding bar graphs showing abundance of populations. (e) scCoGAPS mapping of cord blood CD34⁺ cells (CB HSC) to the EHT dataset and violin plot showing pattern weights. (f) scCoGAPS mapping of the human CS 13 dorsal aorta dataset on the EHT dataset, with plot showing colocalization of populations. (g) Pie charts showing abundance of each cell type mapping from the human CS 13 dataset onto HE, EHT or HSC-like cell types, according to the scCoGAPS analysis in FIG. 6, f.

FIG. 7. Validation of the hematopoietic potential of HE and EHT cells. (a-c) Sorted HE and EHT cells were subcultured for 6 days (representative of n=5). The levels of CD43 and CD34 markers (a) and the levels of CD43, GPA and CD45 markers (c) were assessed at subculture days 3 and 6. (b) Representative pictures of the wells were taken every day during HE and EHT subculture. Scale bars, 100 μm. (d) Expression of globin genes in HSC-like cells assessed by scRNAseq.

FIG. 8. Glycolysis is essential for hematopoietic specification. (a) Representative assay data shows the extracellular acidification rate (ECAR) measured in HE and EHT cells under basal conditions as well as after the addition of the indicated compounds. Bar graphs are shown in FIG. 2, a. (b) Dot plots showing gene expression levels of glycolytic enzymes detected in human CS 13 AGM region (data from Zeng et al.), by mapping of their scRNAseq data onto our dataset (as shown in FIG. 1, c) and based on percent expressed (size of the dots) and average level of expression (color intensity). (c) Glucose is broken down through glycolysis and the resulting pyruvate gives rise to lactate or converts to acetyl-CoA for integration into the TCA cycle. 2-Deoxy-D-glucose (2-DG) blocks the glycolytic flux. (d) Subculture day 3 CD43⁺ cell frequencies±s.e.m. relative to the control are shown (n=7, paired t-test). (e) Subculture day 3 CD43⁺GPA⁺ and subculture day 6 CD43⁺CD45⁺ cell frequencies f s.e.m. relative to the control are presented (n=6 and n=5, respectively, paired t-tests). (f) Subculture day 3 CellTrace Violet (CTV) fluorescence was assessed by flow cytometry and median MFI values are shown (n=4, paired t-tests).

FIG. 9. OXPHOS is increased during EHT even in the absence of glucose. (a) Representative assay data shows oxygen consumption rate (OCR) measured in HE and EHT cells under basal conditions as well as after the addition of the indicated compounds. Bar graphs are shown in FIG. 2, g. (b) Heatmap showing scRNAseq data of OXPHOS-related genes expressed in HE, EHT and HSC-like populations. (c) Heatmap showing scRNAseq data of OXPHOS-related genes expressed in human CS 13 AGM region (data from Zeng et al.), by mapping of their scRNAseq data onto our dataset (as shown in FIG. 1, c). Note that more OXPHOS-related genes are detectable in this primary cell dataset. (d) Dotplot showing scRNAseq data of TCA cycle enzymes expressed in human CS 13 AGM region (data from Zeng et al.), by mapping of their scRNAseq data onto our dataset (as shown in FIG. 1, c).

FIG. 10. Pyruvate catabolism directs hematopoietic lineage specification. (a) FACS-sorted HE, EHT or HSC-like cells were subcultured with or without UK5099 (10 μM). Subculture day 3 CD43⁺/GPA⁺ cell frequencies±s.e.m. relative to the controls for all populations are presented (HE, n=5; EHT, n=6; HSC-like, n=4; paired t-tests). (b) FACS-sorted HE cells were subcultured with or without 1-AA (4 mM). Subculture day 3 CD43⁺GPA⁺ cell frequencies±s.e.m. relative to the control are shown (n=4, paired t-test). (c) Fold change of expression of MPC1 and MPC2 relative to HPRT in shRNA-transduced cells compared to shScrambled (shScr) are shown (n=3, unpaired t tests). Untr, untransduced. (d) HE cells were transduced with shScrambled (shScr), shMPC1, shMPC2 or both the day after the sort and day 3 CD43⁺/GPA⁺ cell frequencies±s.e.m. relative to shScr are presented (n=4; one-way ANOVA test). Untr, untransduced. (e) FACS-sorted HE cells were stained with CTV and fluorescence was assessed by flow cytometry for GPA+ cells at day 3 of subculture with or without UK5099 (10 μM). Representative of n=3. (f-g) FACS-sorted HE, EHT or HSC-like cells were subcultured with or without UK5099 (10 μM). Subculture day 6 CD43⁺ (f) and CD43⁺CD45⁺ (g) cell frequencies±s.e.m. relative to the control for all populations are presented (HE, n=7; EHT, n=7; HSC-like, n=4; paired t-tests). (h) FACS-sorted HE cells were subcultured with or without 1-AA (4 mM). Subculture day 6 CD43⁺ and CD43⁺CD45⁺ cell frequencies±s.e.m. relative to the control are shown (n=3, paired t-test). (i-j) FACS-sorted HE cells were subcultured for 3 days with or without UK5099 (10 μM). CTV for HE-derived CD45⁺ cells (i) and HE-derived HSC-like cell frequencies±s.e.m. relative to the control (n=7, paired t-test) (j) are shown. (k-p) FACS-sorted HE and EHT cells were subcultured with or without DCA (3 mM). Subculture day 3 (k, n=3) and day 6 (1, HE, n=5; EHT, n=4) CD43+GPA+ cell frequencies±s.e.m. relative to the controls are shown (paired t-test). (m) CTV for HE-derived GPA+ cells at subculture day 3 is shown. Representative of n=3. (n) Subculture day 6 CD43⁺CD45⁺ cell frequencies±s.e.m. relative to the controls for both populations are shown (HE, n=5; EHT, n=4; paired t-tests). (o) Fold change of expression of PDK1, PDK2, PDK3 and PDK4 relative to HPRT1 in shRNA-transduced cells compared to shScrambled (shScr) are shown (n=3, paired t tests). (p) HE cells were transduced with shScrambled (shScr) or shPDK1, shPDK2, shPDK3 or shPDK4 the day after the sort and day 6 CD43⁺/CD45⁺ cell frequencies±SEM relative to shScr are presented (n=3; one-way ANOVA tests). (q) CTV for HE-derived CD45⁺ cells are shown. Representative of n=3. (r) HE-derived HSC-like cell frequencies±s.e.m. relative to the control (n=4, paired t-test) are shown. (s) EdU incorporation into HE cells was assessed by flow cytometry after a 24 h pulse at days and 2 of subculture with or without UK5099 (10 μM) or DCA (3 mM) (n=3). (t-u) Percentages of CFU assay colony types obtained from HE cells subcultured with the indicated compounds for 3 days (u) (n=3, 2-way ANOVA test) or 6 days (t) (n=5, 2-way ANOVA test). CFU, colony forming unit; E, erythroid; M, macrophage; G, granulocyte; GEMM, mixed. (v) EryD and EryP CFU-Es obtained from HE cells subcultured with the indicated compounds for 3 days. Scale bars, 100 μm. (w) Fold change in the expression of HBAJ-2 transcripts normalized to KLFJ in CFUs obtained from HE cells treated with UK5099 (10 μM) or DCA (3 mM) relative to non-treated cells. (x) Plots showing percentages of CD45⁺CD56⁺ cells obtained following 35-day co-culture of 3-day subcultured HE cells with OP9-DL1 stroma. During the 3-day subculture, HE cells were treated with the indicated compounds. ns, not significant, *p<0.05, **p<0.01, ***p<0.001, ****p<0.0001.

FIG. 11. Modulation of pyruvate metabolism affects lineage specification in vivo. (a) Pregnant mice were injected with UK5099 or DCA at E9.5 and fetal livers were analyzed at E14.5 by flow cytometry. FL, fetal liver. Levels of HPC-1, HPC-2 (b) and erythroid progenitors (c) as percentages in fetal liver are shown for control (n=10), UK5099-treated (n=14) and DCA-treated (n=16) conditions (one-way ANOVA test). (d) Erythroid differentiation stages according to CD71/Ter119 staining are shown on the control plot. Representative plots showing percentages of cells in each stage are shown for control, UK5099- or DCA-treated conditions. (e) Gating strategy is depicted for sorting LT-HSCs in E14.5 embryos. (f) Percentages of colonies obtained from sorted LT-HSCs are shown for control (n=4), UK5099-treated (n=8) and DCA-treated (n=10) conditions (one-way ANOVA test). (g) Sorted HE cells were kept in co-culture with OP9-DL1 stroma for 3 days, with or without DCA (3 mM) before transplantation into irradiated NSG mice together with bone marrow (BM) support cells. Human cells in peripheral blood (PB) or BM were assessed on weeks 4, 8 or 12. (h) Engraftment levels in PB as percentages of huCD45+ cells are shown (Control, n=6; DCA, n=7). (i) Thymi were harvested on week 12 after transplantation and representative plots showing CD4/CD8 expressing cells are presented for control and DCA-treated conditions. (j) Representative plots and percentages±s.e.m. of CD19+ cells (B cells) in huCD45⁺ cells from PB at week 8 are shown (Control, n=6; DCA, n=6; unpaired t test). (k) Percentages±s.e.m. of human HSCs in huCD45+ cells from the BM are shown (Control, n=6; DCA, n=7; unpaired t tests). (l) Pregnant mice were injected with DCA at E8.5 and AGM regions of embryos at E10.5 were dissected, stained with anti-cKit/anti-CD45 antibodies and analyzed by flow cytometry. (m) Numbers±s.e.m. of cKit⁺CD45⁺ cells in the AGM region of control or DCA-treated embryos are shown. (Control, n=9; DCA, n=10; from 2 independent experiments). ns, not significant, *p<0.05, **p<0.01, ***p<0.001, ****p<0.0001.

FIG. 12. Expression of endothelial and hematopoietic genes in differentiating HE cells. Single-cell RNAseq was performed on control, UK5099-treated and DCA-treated HE cells at day 2 of subculture. Feature plots showing the expression of endothelial (a) or hematopoietic (b) genes on the UMAP in FIG. 4, a. (c) 10×10 dot plots showing the percentages of cells belonging to clusters 6 and 7 in each condition. (d) Numbers of GPA+ clones obtained from single HE cells co-cultured on OP9-DL1 stroma, treated with the indicated compounds for 14 days (n=6 independent experiments, with a total of 552 wells screened for each condition).

FIG. 13. Mechanistic analyses of pyruvate catabolism during EHT. (a) Fold change of expression of LSD1 relative to HPRT1 in shRNA-transduced cells compared to shScrambled (shScr) are shown (n=3, unpaired t tests). Untr, untransduced. (b-c) FACS-sorted HE cells were subcultured with TCP (300 nM), DCA (3 mM) or both. Day 6 CD43⁺CD45⁺ cell frequencies±SEM (b) and Day 6 CD43⁺CD45⁺CD33⁺CD11b⁺ cell frequencies±SEM (c) relative to the control are shown (n=5, one-way ANOVA test). (d) Dot plots showing gene expression levels of cholesterol efflux pathway genes detected by scRNAseq and based on percent expressed (size of the dots) and average level of expression (Color intensity). ns, not significant, *p<0.05, **p<0.01.

FIG. 14. Flow chart showing a method for the production and/or generation of a hematopoietic cell.

FIG. 15. Testing a higher concentration of DCA on NK cell output. (A) Percentage of CD56⁺CD3⁻ cells (total NK cells) shows a marginal increase of CD56⁺CD3⁻ cells compared with the control when treated with DCA at 10 mM. (B) Percentage of CD56⁺CD3⁻ CD16⁺ cells (activated NK cells) increases compared with control when treated with DCA at 10 mM. (C) A co-culture with K562 cells (which are of the erythroleukemia type and represent a target cancer cell for the activated NK cells in the assay) demonstrates a depletion in the percentage of activated NK cells (compared with the condition lacking K562 cells in FIG. 15B), due to the anti-tumour activity of the NK cells.

FIG. 16. The effect of various metabolic regulators on the induction of activated NK cells in the presence and absence of an exemplary target cancer cell (K562). Gating is done on CD56⁺CD3⁻CD16⁺ cells that also express CD107, which is a marker of NK cell activation and cytotoxic degranulation. (A) iPSC-CD34⁺ control that has not been treated with a metabolic regulator; (B) iPSC-CD34⁺ treated with DCA at 10 mM; (C) iPSC-CD34⁺ treated with CP; and (D) iPSC-CD34⁺ treated with cholesterol. The percentage of CD56⁺CD3⁻CD16⁺CD107⁻ cells in the population is shown in the absence (top panel) and presence (bottom panel) of K562 cells. Basal degranulation was higher than ctrl for DCA, CP and Cholesterol treated cells. Co-culture with K562 led to increased degranulation except for Cholesterol treated cells.

### DETAILED DESCRIPTION OF THE PREFERRED EMBODIMENT

Examples disclosed herein relate to methods, compositions, systems, and apparatuses for modulating cell differentiation toward specific lineages, such as specific hematopoietic lineages. The examples disclosed herein are not limited to cells matured to a particular lineage, the technologies disclosed herein may be broadly applicable to different cells and tissues.

During embryonic development, hematopoiesis occurs through primitive and definitive waves, giving rise to distinct blood lineages. Hematopoietic stem cells (HSCs) emerge from hemogenic endothelial (HE) cells, through endothelial to hematopoietic transition (EHT). In the adult, HSC quiescence, maintenance and differentiation are closely linked to changes in metabolism. However, as will be described further below, the emergence of blood may be regulated by multiple metabolic pathways that induce or modulate the differentiation towards specific hematopoietic lineages during human EHT. In both in vitro and in vivo settings, steering pyruvate use towards glycolysis or OXPHOS differentially skews the hematopoietic output of HE cells towards either an erythroid fate with primitive phenotype, or a definitive lymphoid fate, respectively. In certain examples, glycolysis-mediated differentiation of HE towards primitive erythroid hematopoiesis may be dependent on the epigenetic regulator LSD1. In examples, OXPHOS-mediated differentiation of HE towards definitive hematopoiesis may be dependent on cholesterol metabolism. As will be understood by one of skill in the art and explained further below, during EHT, metabolism may be a major regulator of primitive versus definitive hematopoietic differentiation.

Metabolic regulators that may cause preferential use of pyruvate by glycolysis include inhibitors of mitochondrial pyruvate carrier (MPC) (e.g., UK5099, also known as 2-Cyano-3-(1-phenyl-1H-indol-3-yl)-2-propenoic acid and PF-1005023), inhibitors of pyruvate dehydrogenase complex (PDH) (1-AA, i.e. aminoethylphosphinic acid), and shRNA inhibitors of MPC. UK5099 may be used at a concentration of 10 μM but may also be used at concentrations of 1 μM, 5 μM, 20 μM, 50 μM or 100 μM. 1 AA may be used at a concentration of 4 mM but may also be used at concentrations of 0.1 mM, 0.5 mM, 1 mM, 10 mM, 20 mM or 50 mM.

Metabolic regulators that may cause preferential use of pyruvate by oxidative phosphorylation include dichloroacetate (DCA) and shRNA inhibitors of pyruvate dehydrogenase kinases (PDK). DCA may be used at a concentration of 3 mM but may also be used at a concentration of 0.1 mM, 0.5 mM, 1 mM, 5 mM, 10 mM or 30 mM (or any range between these concentrations). For example, the DCA may be used at a concentration ranging from 0.1 mM to 30 mM, such as from 1 mM to 10 mM, or from 3 mM to 10 mM. In some embodiments, the metabolic regulator is CP, which may be used at a concentration of 2.5 μM but may also be used at a concentration of 1 μM, 1.5 μM, 2 μM, 3 μM, 3.5 μM, 4 μM, 4.5 μM or 5 μM (or any range between these concentrations). For example, the CP may be used at a concentration ranging from 1 μM to 5 μM, such as from 1.5 μM to 3.5 μM, or 2 μM to 3 μM. In some embodiments, the metabolic regulator is cholesterol, which may be used at a 0.5× concentration of a cholesterol lipid concentration, such as provided by Thermo Fisher 12531018 (250×), but may also be used at a 0.1×, 0.2×, 0.3×, 0.4×, 0.6× 0.7×, 0.8×, 0.9× or 1× concentration (or any range between these concentrations). For example, the cholesterol may be used at a concentration range from 0.1× to 1×, such as from 0.2× to 0.8×, or 0.4× to 0.6×.

Metabolic regulators that may cause preferential use of oxidative phosphorylation via glutamine metabolism pathway include derivatives of alpha-ketoglutarate such as dimethyl-α-ketoglutarate. Dimethyl-α-ketoglutarate may be used at a concentration of 1 mM but may also be used at concentrations of 0.1 mM, 0.5 mM, 1.5 mM, 2 mM, 5 mM or 10 mM (or any range between these concentrations). For example, the dimethyl-α-ketoglutarate may be used at a concentration ranging from 0.1 mM to 10 mM, such as from 1 mM to 10 mM, from 1 mM to 5 mM, or from 2 mM to 5 mM.

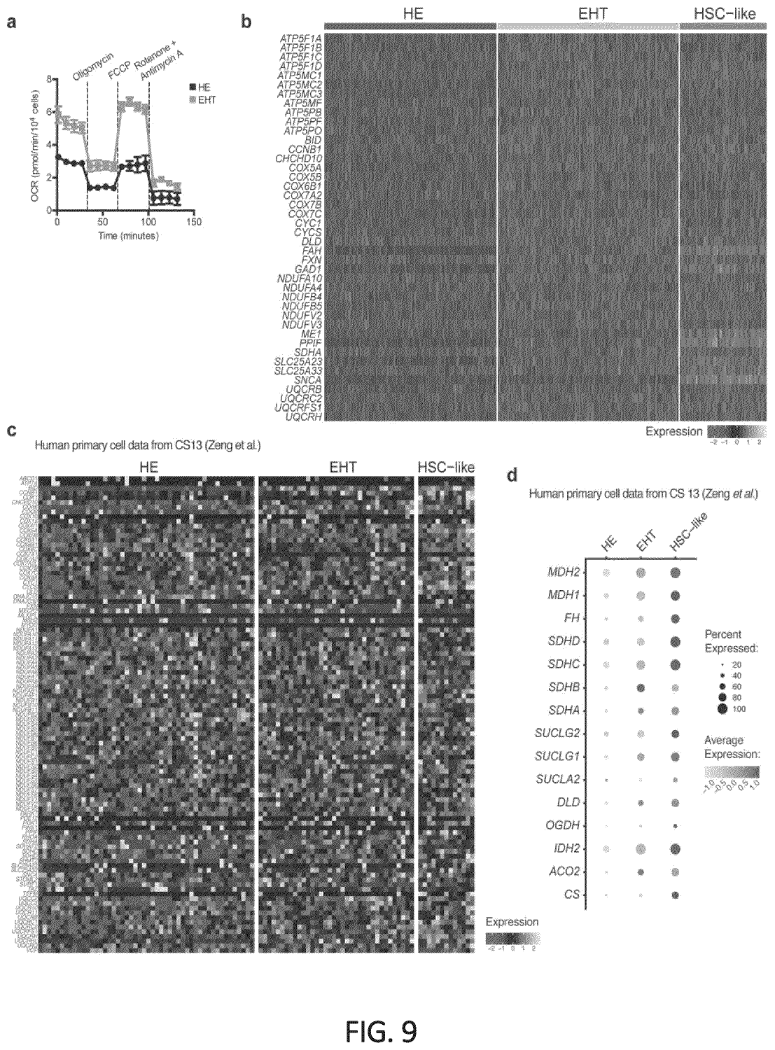

Techniques for determining changes in metabolic pathways are known to the skilled person, such as those described in the Examples herein. For example, promoting OXPHOS may be assessed based on an increased expression of at least one OXPHOS-related gene compared with a control, for example as per FIG. 9. Alternatively, tricarboxylic acid (TCA) cycle activity may be assessed based on an increased expression of at least one TCA cycle enzyme compared with a control, for example as per FIG. 9. Accordingly, a hematopoietic cell that has been treated with a metabolic regulator, such as those described herein, can be distinguished from a hematopoietic cell that has not been treated with a metabolic regulator. As a further example, the hematopoietic cell that has been treated with a metabolic regulator will have an increased expression of CD107 on the cell surface of NK cells compared with an untreated control. In some cases, the increase in CD107 expression is at least 2-fold compared with a relevant control, for example at least 3-fold, at least 4-fold or at least 5-fold compared with a relevant control. In some cases, the level of CD107 expression is greater than 5% of an iPSC-CD34⁺ cell population that has been gated for CD3⁻CD56⁺CD16⁺ cells, for example, greater than 6%, 7%, 8%, 90%, 10%, 11%, 12%, 13%, 14%, 15%, 16%, 17% or 18% of such a population, as determined by flow cytometry, in the absence of exposure to a cancer target cell. Alternatively, or additionally, single cell RNA sequencing can be used to establish alterations in metabolic pathways that derive from the treatment of metabolic regulators, as demonstrated in the Examples. Accordingly, one can perform single cell RNA sequencing on a hematopoietic cell to determine whether it has been exposed to a particular metabolic regulator. Alternatively, or additionally, the anti-tumour activity of the NK cells derived from a source cell treated with a metabolic regulator may be compared with control NK cells (i.e. derived from source cells untreated with a metabolic regulator) in a co-culture assay of the NK cells and a target tumour cells (e.g. K562 cells). A decrease in the total number of tumour cells compared with the control NK cell condition indicates increased anti-tumour activity.

Techniques for modifying hematopoietic cells to express a chimeric antigen receptor (CAR) are known in the art. The CAR may be introduced to the source cell prior to, during, and/or after treatment with the metabolic regulator. Preferably, the CAR may be introduced to the source cell (e.g. iPSC-CD34⁺) prior to treatment with a metabolic regulator, as the CAR expression is then passed down to daughter cells that derive from the source cell following cell expansion. Therefore, one can obtain CAR-NK cells that have been enhanced by the metabolic regulator treatment. Alternatively, the CAR may be introduced following enrichment of NK cells that derive from the source cells treated with a metabolic regulator. Alternatively, the CAR may be introduced at an intermediate stage between the aforementioned.

As demonstrated in the examples below, in embodiments during EHT, transitioning cells go through substantial changes in energy use and metabolism, with simultaneous increases in glycolysis and TCA cycle/OXPHOS. In certain examples, glucose may play a role in both glycolysis and the TCA cycle, and blocking its use with 2-DG may impair hematopoietic differentiation of HE cells. In quiescent HSCs, glycolysis may be regulated by hypoxia through the stabilization of hypoxia-inducible factor-1α (HIF-1α). In certain embodiments, the transition from HE to HSCs may also be regulated by HIF-1α. HIF1α may be a regulator of hematopoietic progenitor and stem cell development in hypoxic sites of the mouse embryo. Therefore, as will be understood by one of skill in the art, in embodiments, HIF-1α-dependent induction of glycolysis may be required for EHT.

In certain embodiments, glycolysis may be sufficient to provide energy for primitive hematopoiesis. At early embryonic stages, oxygen is not systemically available, and glycolysis may be the pathway of choice to produce energy. In developing embryos, primitive erythroid cells may perform high rates of glycolysis to fuel their rapid proliferation. In some embodiments, boosting glycolysis by blocking pyruvate entry into the mitochondria redirects HE differentiation towards primitive erythropoiesis at a very early stage of EHT, as shown by an increased frequency of erythroid transcription factor-expressing cells at the single cell level as well as higher levels of erythroid factors and embryonic/fetal-specific globins.

The examples below indicate a role for the TCA cycle and OXPHOS in preferentially inducing definitive hematopoietic identity. For example, fueling the TCA cycle with DCA treatment may lead to an increased differentiation of HE cells toward a definitive CD45⁺ lineage. While PDK inhibition with DCA may not affect primitive erythroid cell formation, it may induce definitive hematopoiesis, as measured by increased lymphoid lineage biases which we have shown both in vitro and in vivo. In embodiments, DCA-treatment of HE cells leads to an increased lymphoid reconstitution including T cells in NSG mice, therefore pyruvate may be able to not only modulate erythroid and lymphoid lineage outputs but also primitive and definitive states of HE-derived cells. In certain examples, DCA promotes Notch1-dependent CD45+ cell formation by fueling cholesterol biosynthesis in HE cells. Moreover, the accumulation of cholesterol in HE cells may lead to an increase in the expression of cholesterol efflux genes. Indeed, cholesterol efflux mechanisms have been previously shown to regulate HSPC proliferation. In certain embodiments, ATP-binding cassette transporters and HDL suppress hematopoietic stem cell proliferation and therefore HE cells transitioning to become HSPCs may express higher levels of cholesterol efflux genes. Consequently, as will be understood by one of skill in the art, a direct metabolic change in HE cells, namely increased acetyl-CoA content, can promote cholesterol metabolism and control definitive hematopoietic output.

Distinct EHT cell subsets or pre-HSCs can present different lineage propensities. Considering the examples below, in certain embodiments, metabolism can influence the differentiation of HE cells, suggesting that lineage propensities may be decided at the HE level. In line with this connection, a recent study combining scRNAseq with lentiviral lineage tracing revealed that cell fate biases appear at a much earlier stage during hematopoietic development than previously described with conventional methods. Murine HSCs may present lymphoid or myeloid hematopoietic lineage biases due to epigenetic priming which is established prior to their formation. Linking epigenetic changes to metabolism is a newly emerging field which reconciliates metabolic alterations with transcriptional regulation of cellular processes. In certain embodiments here, we show here that erythroid fate induction by MPC inhibition is dependent on an epigenetic factor, LSD1. As will be understood by one of skill in the art, additional epigenetic modifications that are occurring concomitantly to the metabolic changes in EHT may contribute to specify cell fate.

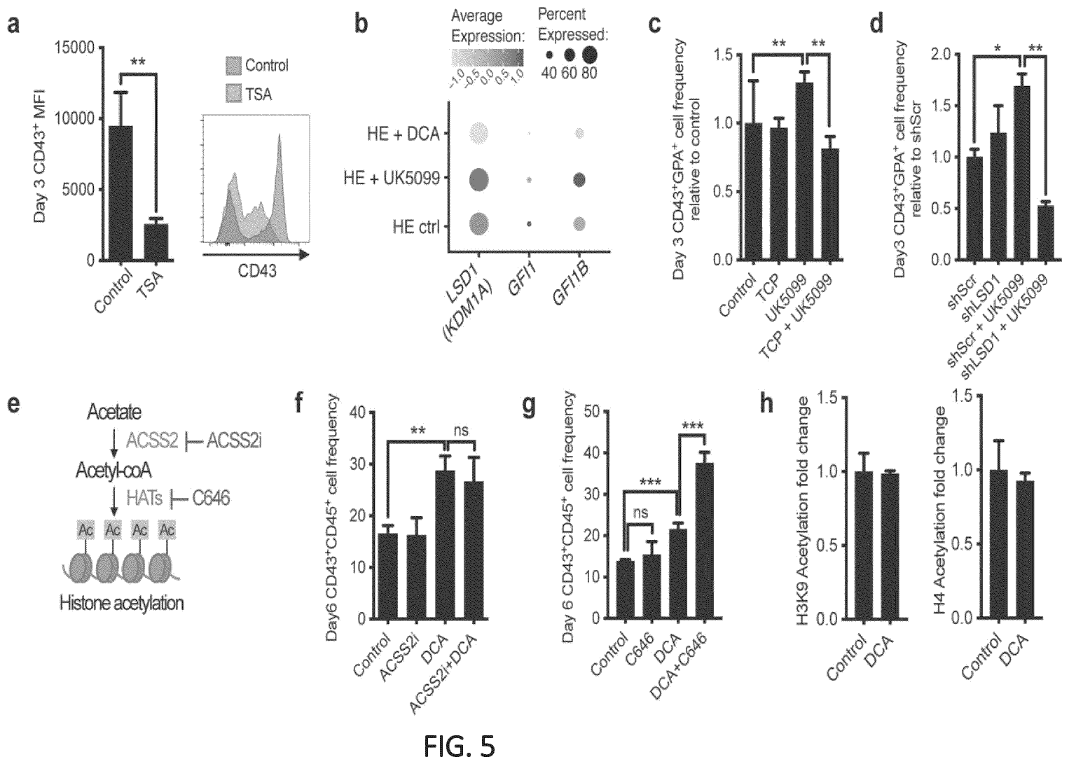

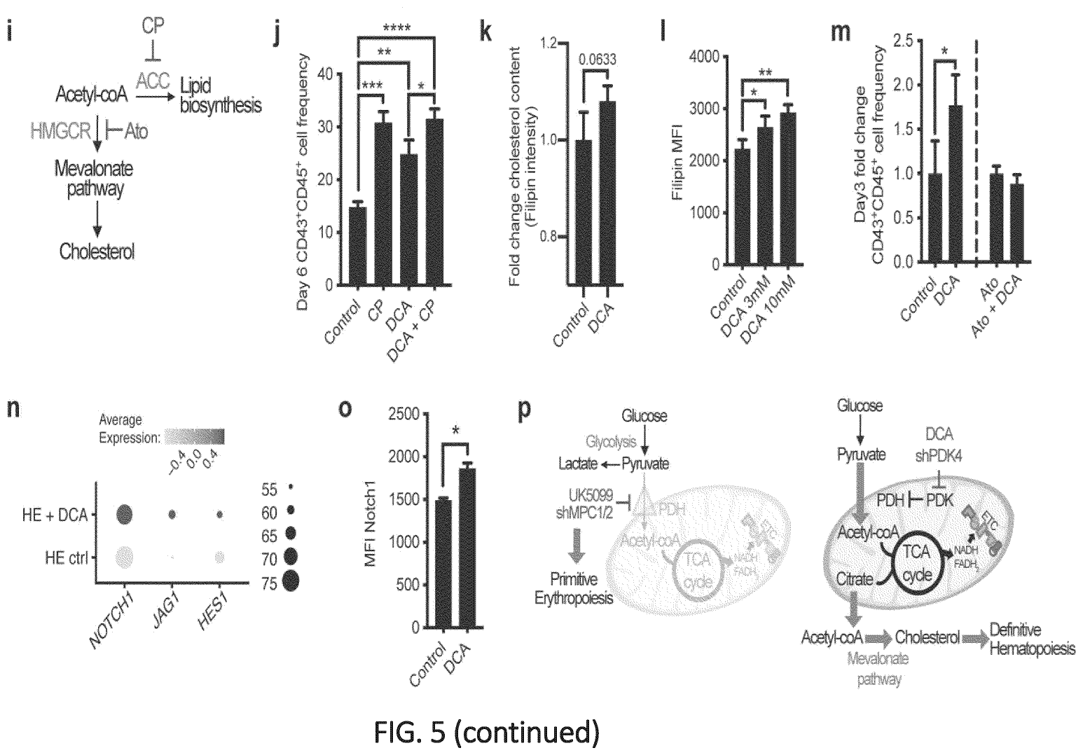

The examples described below indicate that the lineage propensities of primitive and definitive hematopoietic waves may be shaped by nutrient availability in the YS and AGM niches. Due to scarcity of oxygen in early embryonic stages, the primitive hematopoietic wave may depend on glycolysis to form erythroid cells expressing embryonic globins with high affinity for oxygen (FIG. 5, p). This may allow for an efficient distribution of oxygen to newly forming tissues and promotes the use of OXPHOS, which may initiate the emergence of the definitive hematopoietic waves (FIG. 5, p).

In certain embodiments, use of metabolic modulators to direct definitive HSC development in vitro from PSCs may provide a basis to produce transplantable cells, able to reconstitute the hematopoietic system of patients with hematological malignancies and disorders. For example, hematologic malignancies are cancers that affect the blood, bone marrow, and lymph nodes. This classification includes various types of leukemia (acute lymphocytic (ALL), chronic lymphocytic (CLL), acute myeloid (AML), chronic myeloid (CML)), myeloma, and lymphoma (Hodgkin's and non-Hodgkin's (NHL)).

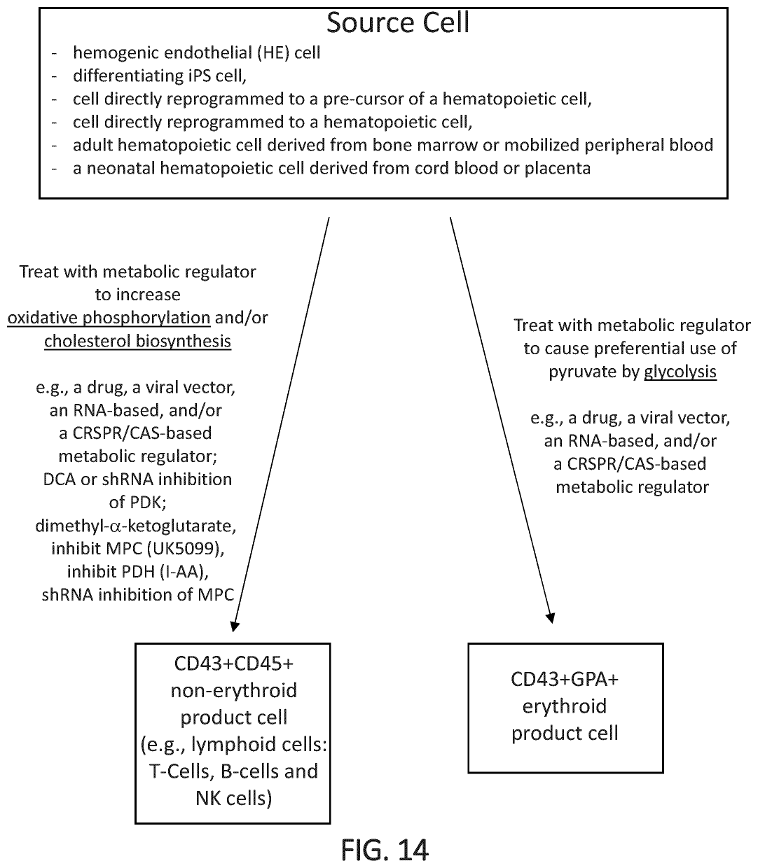

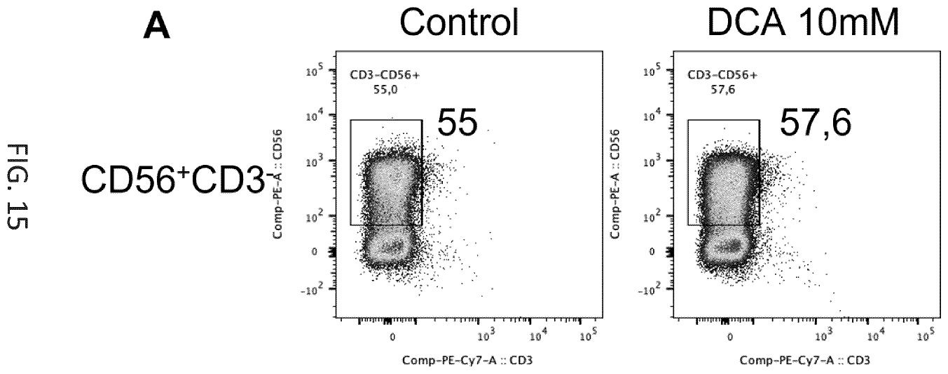

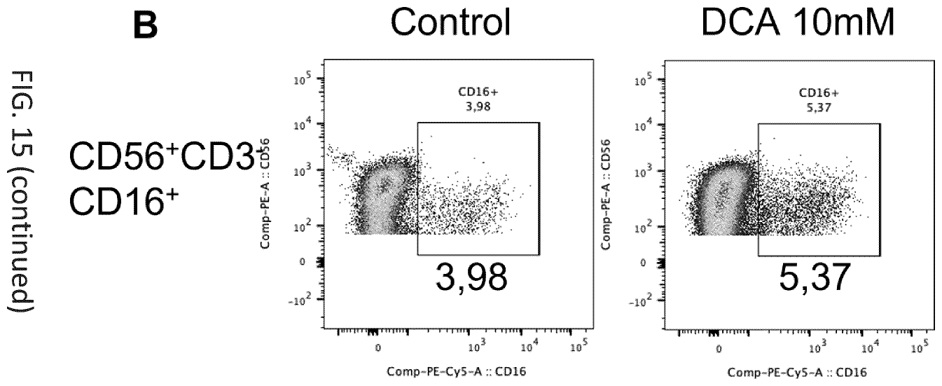

We disclose the production of off-the-shelf hematopoietic cells or derived cells and/or products for therapeutics. FIG. 14 depicts a flow chart showing a method for the production and/or generation of a hematopoietic cell. Such a hematopoietic cell may be used in treatments such as described herein, such as the hematologic malignancies described herein. In certain embodiments, such a method may involve providing a source cell, which may be any of the suitable cell types disclosed herein such as a hemogenic endothelial (HE) cell, an iPS cell (such as a differentiating iPS cell), a cell directly reprogrammed to a pre-cursor of a hematopoietic cell, a cell directly reprogrammed to a hematopoietic cell, an adult hematopoietic cell derived from bone marrow or mobilized peripheral blood and a neonatal hematopoietic cell derived from cord blood or prenatal tissue (e.g. placenta). In some examples, this source cell may then be treated with a metabolic regulator. In certain examples, the metabolic regulator may direct the source cell to utilize pyruvate by glycolysis or oxidative phosphorylation. In examples, the metabolic regulator may be a molecule, a drug, a viral vector, or an RNA-based, and/or a CRISPR/CAS-based metabolic regulator. Once the source cell has been treated by the metabolic regulator, the source cell may differentiate into a CD43⁺GPA⁺ erythroid cell or a CD43⁺CD45⁺ non-erythroid cell or other suitable cell. In embodiments, the CD43⁺CD45⁺ non-erythroid cell may be a lymphoid cell, such as T and/or B cells, NK cells, NKT cells, and/or ILCs.

In certain embodiments, pyruvate may be blocked from entering the mitochondria, thereby reducing tricarboxylic acid (TCA) cycle activity. The metabolic regulator used for blocking may be a mitochondrial pyruvate carrier (MPC) and/or a pyruvate dehydrogenase complex (PDH). UK5099 may be used to inhibit MPC, while 1-AA may be used to inhibit PDH. In some embodiments, the expression of MPC subunits (MPC1 and/or MPC2) is/are downregulated using shRNAs.

In some embodiments, the source cell may be directed to use pyruvate via oxidative phosphorylation (OXPHOS) and the source cell may be differentiated into CD43⁺CD45⁺ non-erythroid cells by definitive hematopoietic differentiation. Such pyruvate use may be blocked by any suitable metabolic regulator disclosed herein. In embodiments, DCA or a related molecule or an shRNA may be used to inhibit pyruvate dehydrogenase kinases (PDKs) and thereby promote pyruvate use by OXPHOS. Pyruvate flux into the mitochondria may then be increased, thereby amplifying acetyl-CoA production and in turn promoting cholesterol metabolism and favoring non-erythroid hematopoietic output. Dimethyl α-ketoglutarate (DMK) or a related molecules may also be used to promote glutamine metabolism pathway feeding of the tricarboxylic activity/oxidative phosphorylation.

In embodiments, metabolic regulation of a method for generating hematopoietic calls such as described herein may include inhibitors of the lipid biosynthesis pathway, such as CP-640186 and related molecules.

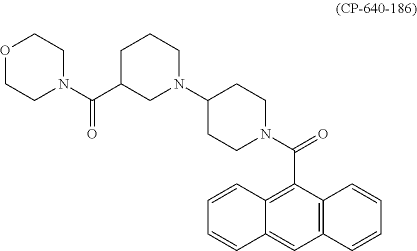 [SMILES: O=C(c1c2ccccc2cc2ccccc12)N1CCC(N2CCCC(C(=O)N3CCOCC3)C2)CC1]

In certain examples, metabolic regulation may include inhibitors of histone acetylation pathway, C646 and related molecules.

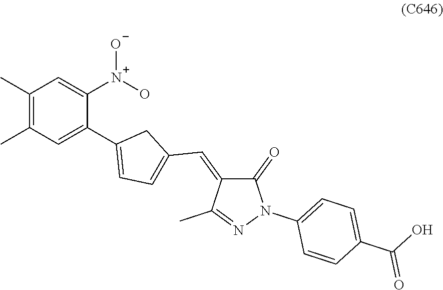 [SMILES: CC1=NN(c2ccc(C(=O)O)cc2)C(=O)/C1=C/C1=CC=C(c2cc(C)c(C)cc2[NH+]([O-])O)C1]

As will be understood by one of skill in the art, a method of reconstituting a hematopoietic system in a subject with a hematological malignancy or disorder such as described herein may involve any of the cells disclosed herein, such as the cells described in relation to FIG. 14, then transplanting such cells into the subject.

### Terminology

Any patents and applications and other references noted herein, including any that may be listed in accompanying filing papers, are incorporated herein by reference. Aspects of the disclosure can be modified, if necessary, to employ the systems, functions, and concepts of the various references described herein to provide yet further implementations.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described herein may be removed, others may be added. For example, the actual steps or order of steps taken in the disclosed processes may differ from those shown in the figure. Depending on the embodiment, certain of the steps described herein may be removed, others may be added. Furthermore, the features and attributes of the specific embodiments disclosed herein may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the description of certain embodiments and may be defined by the claims. The language of the claims is to be interpreted broadly based on the language employed in the claims and not limited to the examples described in the present specification or during the prosecution of the application, which examples are to be construed as non-exclusive.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

Appendix 1 is a publication of Applicants work entitled “Pyruvate metabolism guides definitive lineage specification during hematopoietic emergence”, EMBO Reports, Volume 23, Issue 2: e54384, 3 Feb. 2022 (Published Online Dec. 16, 2021).

Certain embodiments of the disclosure are encompassed in the claims presented at the end of this specification, or in other claims presented at a later date.

The following non-limiting examples serve to illustrate various aspects of the disclosure.

### Example 1—Recapitulation of Human EHT and Hematopoietic Differentiation In Vitro

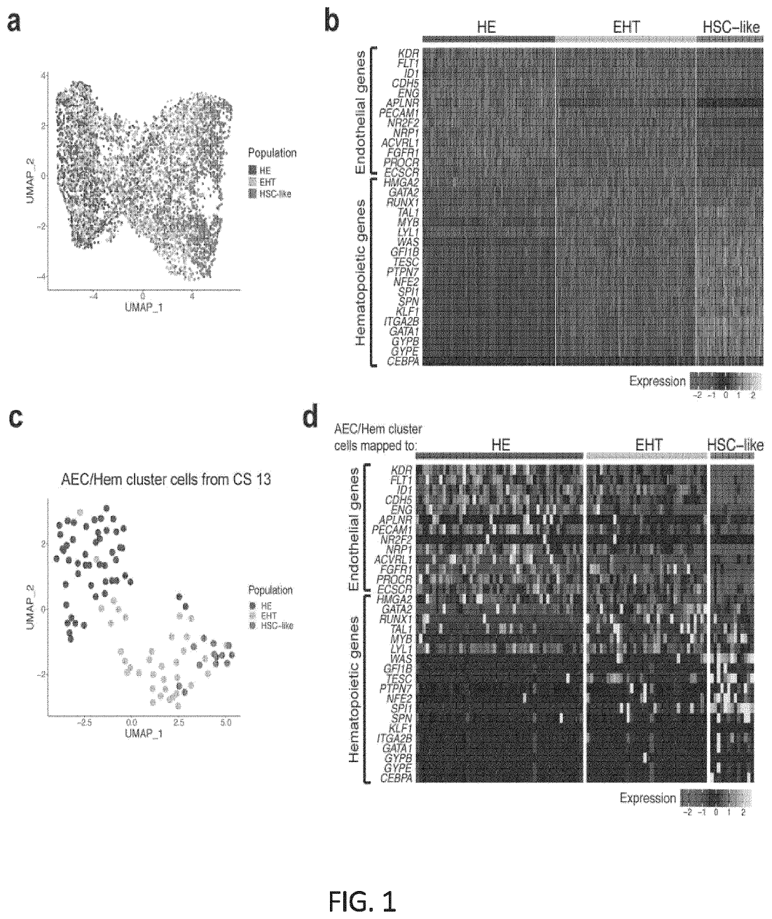

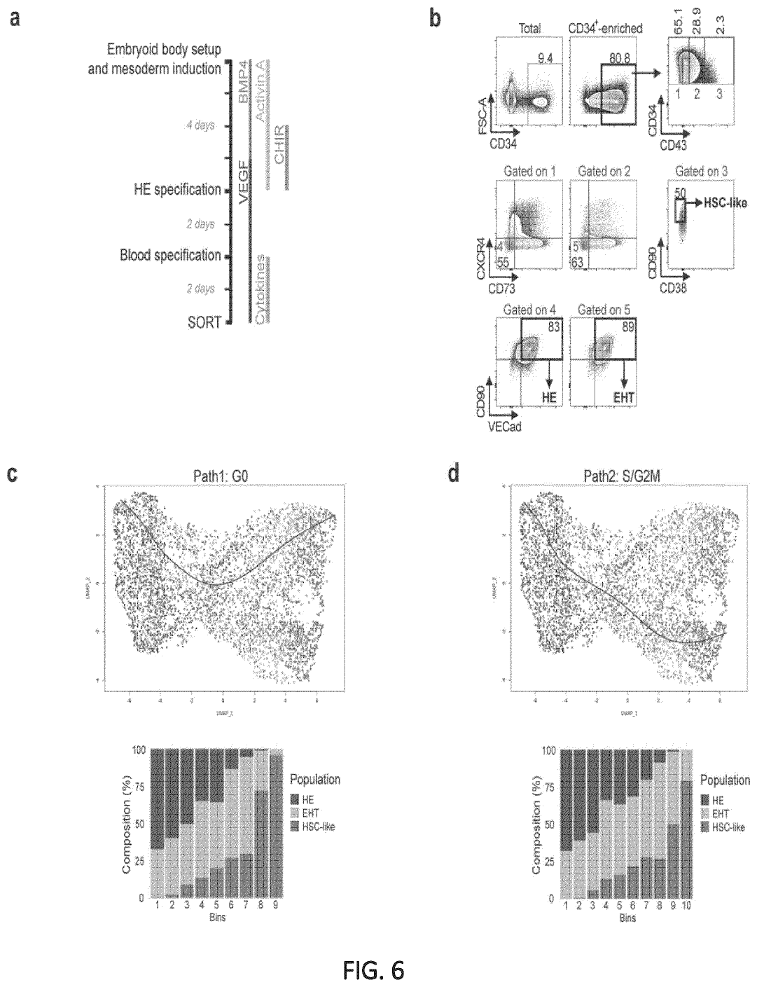

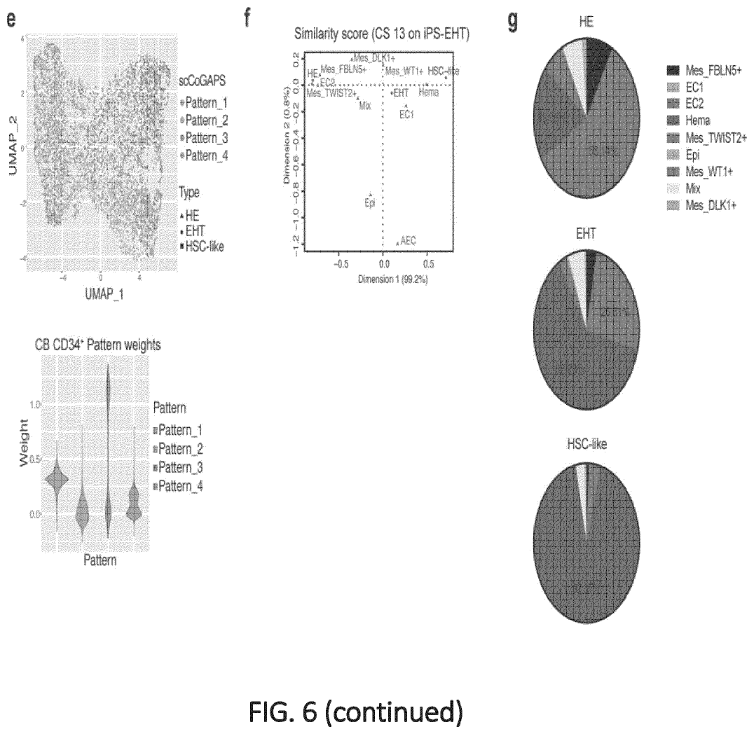

To combine both primitive and definitive hematopoietic waves in cultures, two previously described small molecules were combined during human iPSC differentiation (FIG. 6, a): CHIR99021, a WNT pathway agonist which supports definitive hematopoiesis (Ng, E. S. et al. Differentiation of human embryonic stem cells to HOXA+ hemogenic vasculature that resembles the aorta-gonad-mesonephros. Nature Biotechnology 34, 1168-1179 (2016)) and Activin A, which promotes primitive hematopoiesis (Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012)). After integrating these modifications to a previously described hematopoietic differentiation protocol (Ditadi, A. & Sturgeon, C. M. Directed differentiation of definitive hemogenic endothelium and hematopoietic progenitors from human pluripotent stem cells. Methods 101, 65-72 (2016)), hemogenic endothelial cells (HE) were obtained, such transitioning cells which express CD43 at intermediate levels (EHT) (Guibentif, C. et al. Single-Cell Analysis Identifies Distinct Stages of Human Endothelial to-Hematopoietic Transition. Cell Reports 19, 10-19 (2017)) and hematopoietic stem like cells (HSC-like) (for gating strategies, see FIG. 6, b). The HSC-like cells were termed as such as these cells are immunophenotypically similar to HSCs, but do not possess engraftment potential as demonstrated in previous studies (Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012); Doulatov, S. et al. Induction of multipotential hematopoietic progenitors from human pluripotent stem cells via re-specification of lineage-restricted precursors. Cell Stem Cell 10 3, (2013); Ditadi, A. et al. Human definitive haemogenic endothelium and arterial vascular endothelium represent distinct lineages. Nat Cell Biol 17, 580-591 (2015) and Elcheva, I. et al. Direct induction of haematoendothelial programs in human pluripotent stem cells by transcriptional regulators. Nat Commun 5, 1-11 (2014)). These three populations were characterized transcriptionally using single-cell RNA sequencing (scRNAseq). The UMAP visualization placed the HE cells distally from HSC-like cells, with EHT cells bridging these two populations, confirming the expected sequential EHT process (FIG. 1, a). Additionally, a pseudotime analysis was performed on the differentiation process in our dataset. It was observed that the HSC-like cell population presented both GO and S/G2M states and therefore, to provide a complete analysis, the pseudotime analysis was performed for both GO and S/G2M states (FIG. 6, c, d; respectively). In both cases, an abundance of HE cells was observed at the start, EHT cells in the middle and HSC-like cells at the end of the differentiation trajectory (FIG. 6, c, d; bar graphs), regardless of the cell cycle state of the cells. HE cells expressed endothelial markers such as KDR, FLT1, CDH5 but no hematopoietic markers; in contrast, EHT cells expressed both endothelial and hematopoietic markers and HSC-like cells only expressed hematopoietic markers such as RUNX1, TAL1, WAS and SPN (FIG. 1, b), as shown previously in other EHT systems (Zhou, F. et al. Tracing haematopoietic stem cell formation at single-cell resolution. Nature 533, 487-492 (2016); Swiers, G. et al. Early dynamic fate changes in haemogenic endothelium characterized at the single-cell level. Nat Commun 4, 2924 (2013); and Guibentif, C. et al. Single-Cell Analysis Identifies Distinct Stages of Human Endothelial to-Hematopoietic Transition. Cell Reports 19, 10-19 (2017)). We generated an isolated cord blood CD34⁺ cells dataset and projected it onto our EHT process dataset using the scCoGAPS package and observed that the highest pattern weights were for pattern 1 and part of pattern 3, both encompassing our HSC-like cluster (FIG. 6, e), demonstrating that cord blood CD34+ cells share the most transcripts with our iPSC-derived HSC-like cells. We compared our EHT process data to a recently published scRNAseq analysis of primary human embryonic cells at Carnegie stage (CS) 1336. Of the 99 cells in the arterial endothelial and hematopoietic (AEC/Hem) clusters, 50, 36 and 13 cells mapped to our HE, EHT and HSC-like populations, respectively (FIG. 1, c); and they clustered similarly to our EHT dataset in FIG. 1, a. Furthermore, similarly to our EHT dataset, AEC/Hem cluster cells mapped to HE expressed endothelial markers such as KDR, FLT1, CDH5 and cells mapped to HSC-like cells expressed hematopoietic markers like RUNX1, TAL1, WAS and SPN (FIG. 1, d). To rule out any bias in the data, we mapped the complete human CS 13 dorsal aorta population dataset from Zeng et al. onto our dataset using the scCoGAPS package. We classified the resulting patterns according to the cell types described by Zeng et al. (Table 1) and the resulting similarity scores showed that the CS 13 endothelial cell (EC2) population mapped close to our HE cells and the CS 13 Hema cluster mapped close to both our EHT and HSC-like cells (FIG. 6, f). When we assessed the relative abundance of each cell type mapping to our dataset, we observed that 58.14% of CS 13 cells clustering to our HE cells were endothelial cells (EC2) and 19.38% were hematopoietic cells (FIG. 6, g). In contrast, 62.28% of CS 13 cells mapping to our EHT cells were hematopoietic cells and 25.61% were endothelial cells (EC2). Lastly, almost all cells mapping to our HSC-like cells were hematopoietic cells (92.37%). Thus, our system successfully recapitulates the human EHT process and the HE, EHT and HSC-like populations we obtain possess hemato-endothelial transcriptional signatures equivalent to that of the cell types that occur in the human embryo at CS 13.

TABLE 1
Classification of patterns resulting from scCoGAPS according to cell type

| | AEC | EC | Epi | Hema | Mes_DLK1+ | Mes_FBLN5+ | Mes_TWIST2+ | Mes_WT1+ |
|---|---|---|---|---|---|---|---|---|
| Pattern_1 | 0 | 0 | 0 | 0 | 2 | 40 | 0 | 0 |
| Pattern_10 | 0 | 39 | 0 | 0 | 0 | 0 | 0 | 0 |
| Pattern_2 | 0 | 137 | 0 | 0 | 0 | 0 | 0 | 0 |
| Pattern_3 | 4 | 2 | 0 | 28 | 2 | 0 | 41 | 1 |
| Pattern_4 | 0 | 0 | 0 | 0 | 0 | 0 | 46 | 0 |
| Pattern_5 | 0 | 0 | 38 | 0 | 0 | 0 | 0 | 0 |
| Pattern_6 | 0 | 0 | 0 | 0 | 0 | 0 | 1 | 25 |
| Pattern_7 | 1 | 6 | 0 | 1 | 9 | 1 | 14 | 2 |
| Pattern_8 | 0 | 0 | 0 | 0 | 80 | 0 | 2 | 0 |
| Pattern_9 | 69 | 0 | 0 | 1 | 0 | 0 | 0 | 0 |

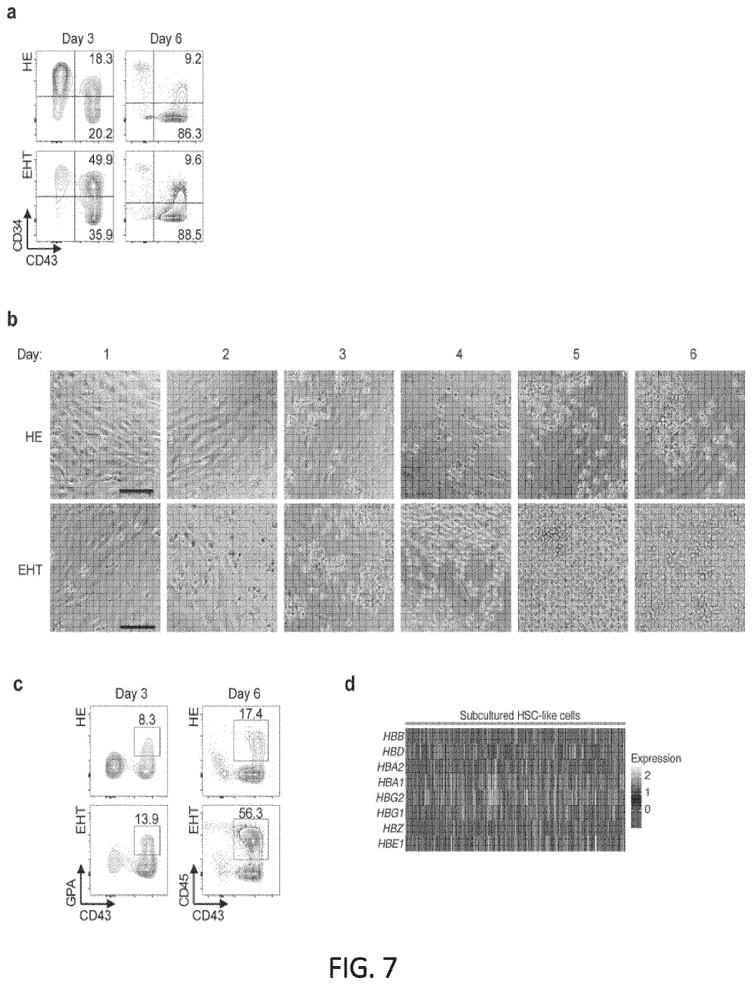

Next, we verified the hematopoietic potential of both HE and EHT populations. Both cell types gave rise to hematopoietic cells (CD43⁺) (FIG. 7, a). At day 6 of subculture, almost all cells (>96%) deriving from HE or EHT cells were CD43⁺ and the majority had lost CD34 expression (>86%), pointing to their maturation. In both cell cultures, spindle-shaped endothelial cells changed their morphology to round hematopoietic cells (FIG. 7, b). In both HE and EHT-derived subcultures, an erythroid (CD43‘GPA’) cell population and a non-erythroid pan-hematopoietic CD43⁺CD45⁺ cell population were clearly discernible at days 3 and 6, respectively (FIG. 7, c). According to the model described by Kennedy et al. (Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012)), the timeframes in which the CD43⁺GPA⁺ and the CD43⁺CD45⁺ cell populations are generated suggest their primitive and definitive natures, respectively. Moreover, the presence of embryonic (HBZ, HBE1), fetal (HBA1, HBA2, HBG1, HBG2) and adult (HBD, HBB) globin upregulation in subcultured HSC-like cells further supports that, in this setting, we obtain both primitive and definitive hematopoietic cells (FIG. 7, d). Altogether, our results show that this differentiation system allows to accurately model the human EHT process and subculturing the resulting HE cells efficiently gives rise to primitive and definitive hematopoietic populations.

### Example 2—Glycolysis Fuels Distinct Processes During EHT

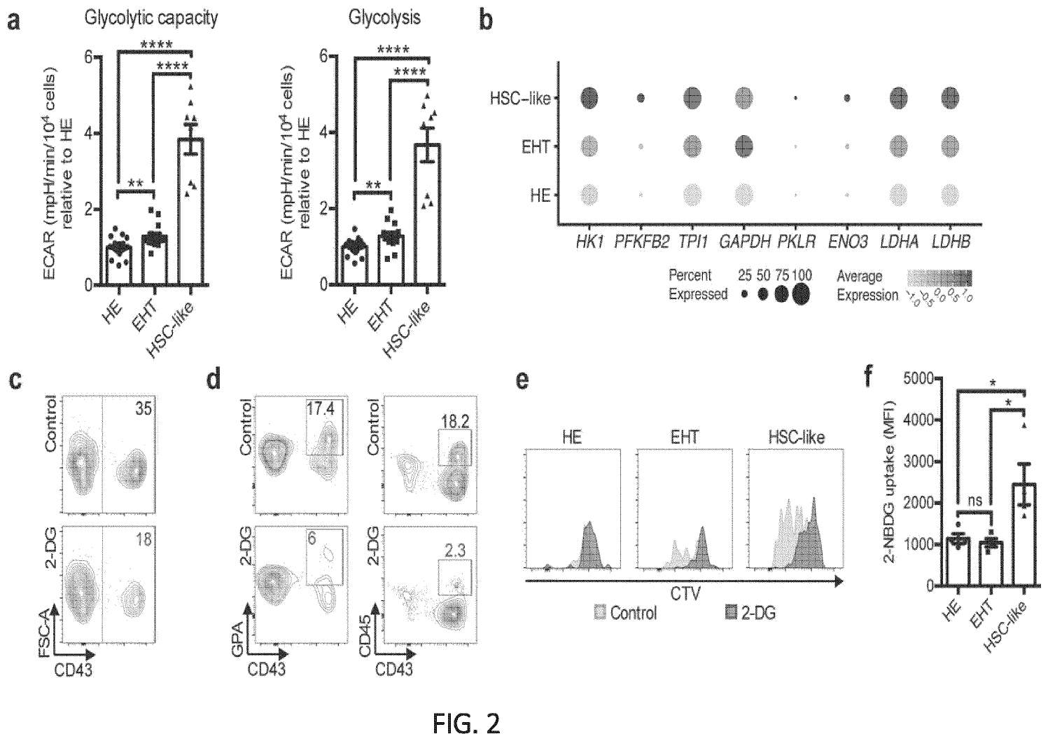

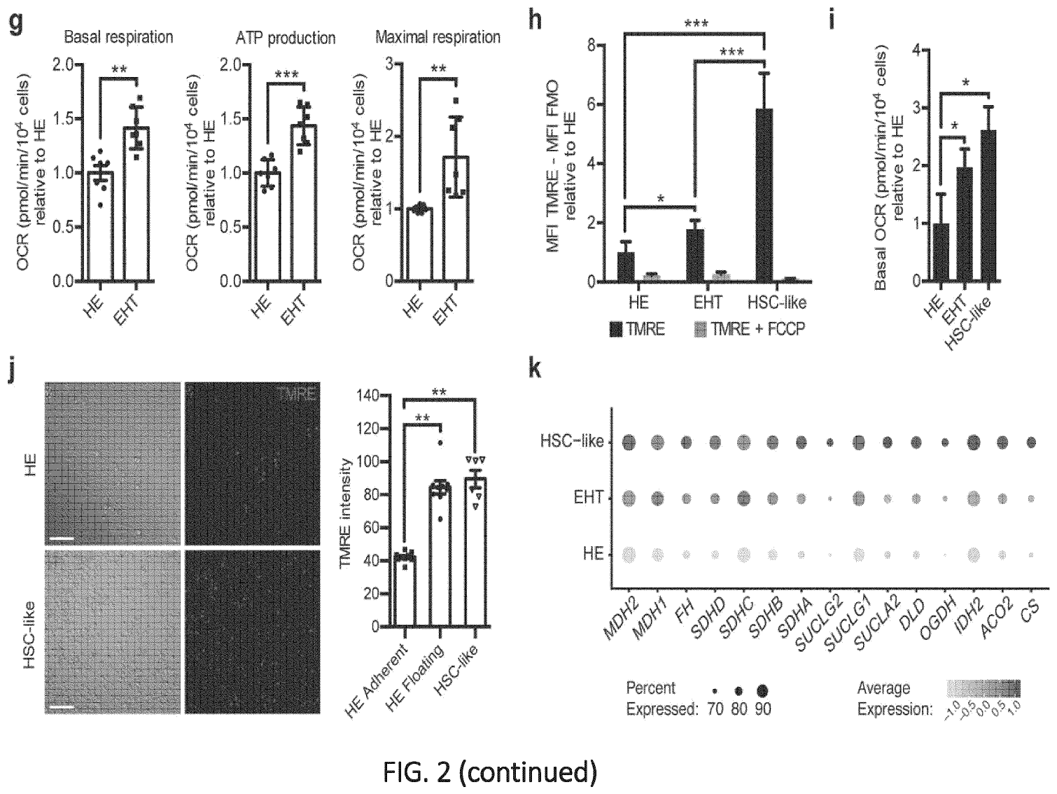

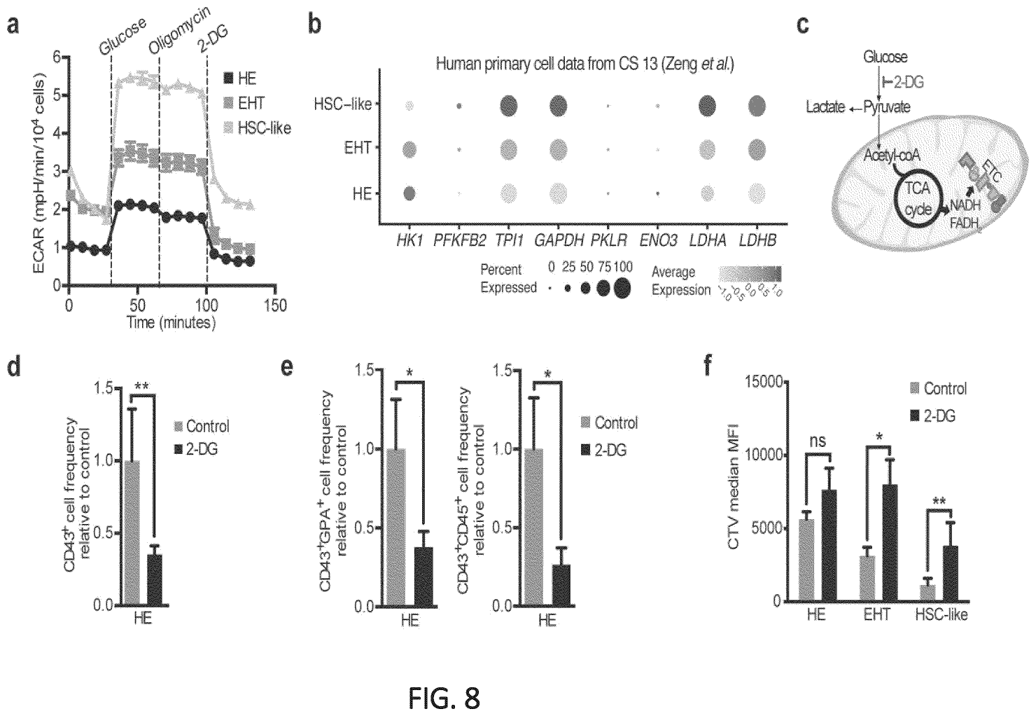

In order to describe the metabolic processes occurring in EHT populations, we first assessed glycolysis in HE, EHT and HSC-like cells. We observed a gradual increase in glycolytic capacity and glycolysis with differentiation (FIG. 2, a, FIG. 8, a). Moreover, expression of the glycolytic enzymes HK1, PFKFB2, TPI1, GAPDH, PKLR, ENO3, LDHA and LDHB as assessed by scRNAseq also increased during EHT (FIG. 2, b). We also observed an increase in the majority of these glycolytic enzymes during EHT in human primary cells from CS 13 (Zeng, Y. et al. Tracing the first hematopoietic stem cell generation in human embryo by single-cell RNA sequencing. Cell Res 1-14 (2019)), confirming our in vitro results (FIG. 8, b).

To understand whether glycolytic activity is required during EHT, we treated HE cells with a glucose analog, 2-Deoxy-D-glucose (2-DG), which blocks glycolysis (FIG. 8, c). This treatment significantly reduced CD43+ cell output from HE cells at day 3 of subculture (FIG. 2, c, FIG. 8, d). Moreover, in the presence of 2-DG, generation of the CD43⁺GPA⁺ cell population at day 3 and the CD43⁺CD45⁺ cell population at day 6 was significantly impaired, dropping to less than 50% of the control (FIG. 2, d, FIG. 8, e). Intriguingly, proliferation rates of EHT or HSC-like cells, but not HE, were significantly reduced in the presence of 2-DG (FIG. 2, e, FIG. 8, f). These results suggest that glycolysis is important for proliferation during the EHT process and as such, the use of glycolysis is required to induce hematopoietic differentiation from HE cells.

### Example 3—Mitochondrial Respiration Gradually Increases During the EHT Process

Along with increased glycolysis and proliferation, HSC-like cells also had increased glucose uptake compared to HE and EHT cells (FIG. 2, f). Interestingly, even though glycolytic flux was higher in EHT cells as compared to HE cells, glucose uptake was comparable in these two cell types. This result prompted us to investigate whether mitochondrial respiration was more active in HE versus EHT cells. Unexpectedly, EHT cells displayed higher levels of basal respiration, ATP production and maximal respiration as compared to HE cells (FIG. 2, g; FIG. 9, a). Moreover, mitochondrial activity measured by TMRE staining was significantly increased in individually analyzed EHT cells compared to HE cells and we observed an even higher rate in the case of HSC-like cells (FIG. 2, h). Treatment with FCCP, which depolarizes mitochondria, abrogated the TMRE signal in all cell types, suggesting that OXPHOS was active in these populations (FIG. 2, h). In line with the TMRE staining, the highest basal respiration rates we detected were in HSC-like cells (FIG. 2, i). Using live cell imaging by confocal microscopy, we compared mitochondrial activity in spindle-shaped HE cells versus their newly-formed round hematopoietic progeny in the same well. The TMRE staining intensity measurements showed a 2-fold higher mitochondrial activity in round as compared to spindle-shaped cells in HE wells and this value was similar to the levels detected in HSC-like cells (FIG. 2, j). Moreover, we observed a gradual increase in the expression of several genes implicated in OXPHOS, including subunits of Complex I (genes termed NDUF), II (SDHA), IV (genes termed COX) and V (genes termed ATP5) in HE, EHT and HSC-like populations by scRNAseq (FIG. 9, b). This result was accompanied by a progressive increase in TCA cycle enzymes during EHT (FIG. 2, k). We observed a gradual increase in both OXPHOS-related genes and TCA cycle enzymes during EHT in human primary cells at CS 13 (Zeng, Y. et al. Tracing the first hematopoietic stem cell generation in human embryo by single-cell RNA sequencing. Cell Res 1-14 (2019)), confirming our in vitro findings (FIG. 9, c, d). Taken together, these results show that TCA cycle activity, mitochondrial respiration and OXPHOS gradually increase during the EHT process.

### Example 4—Modulation of Pyruvate Use Biases HE Towards Differing Hematopoietic Lineage Outputs

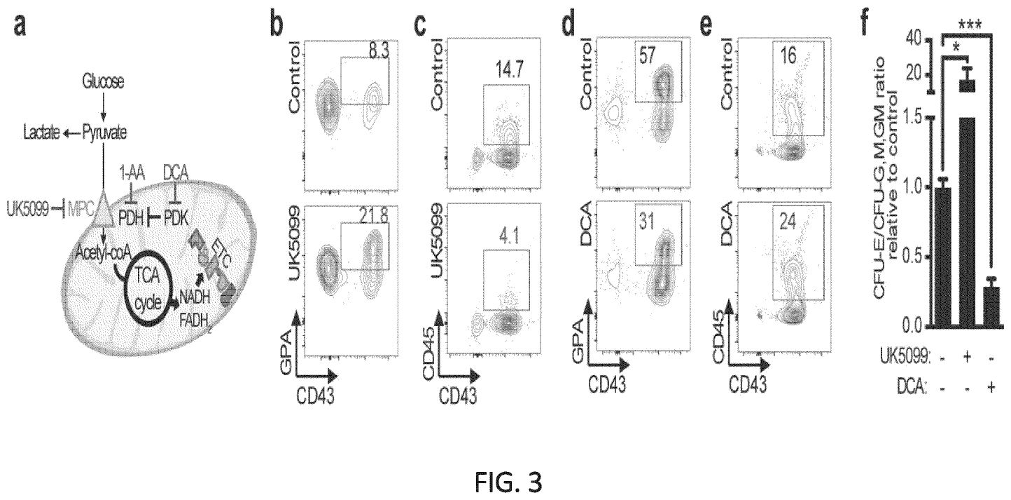

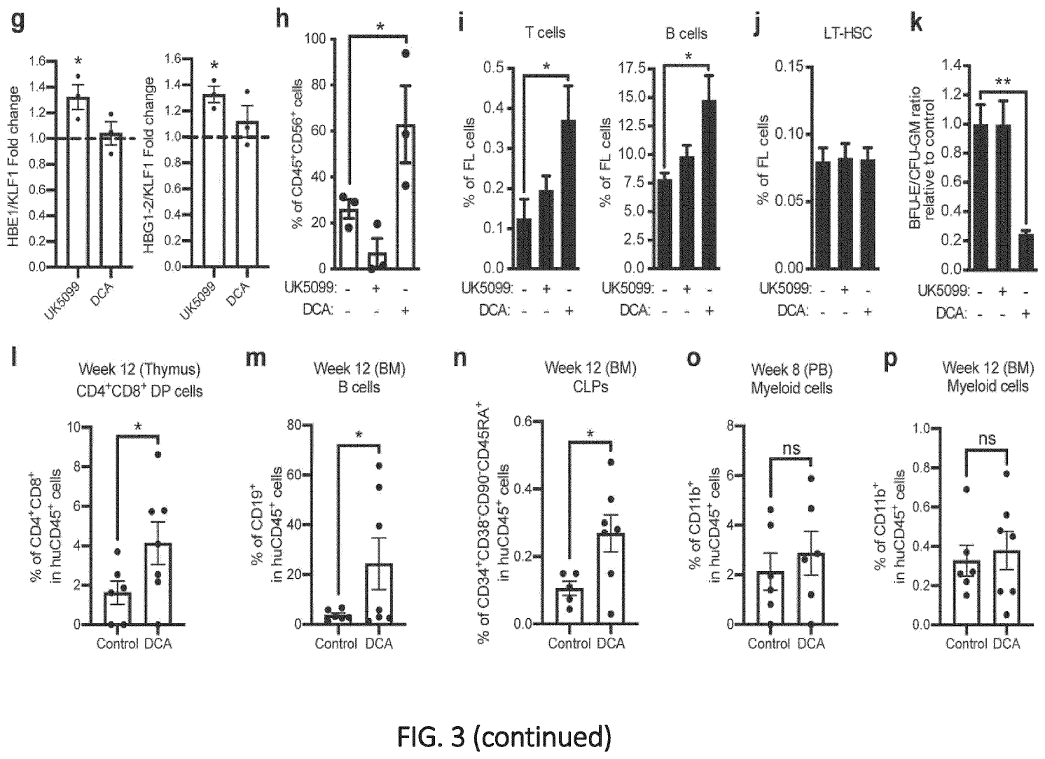

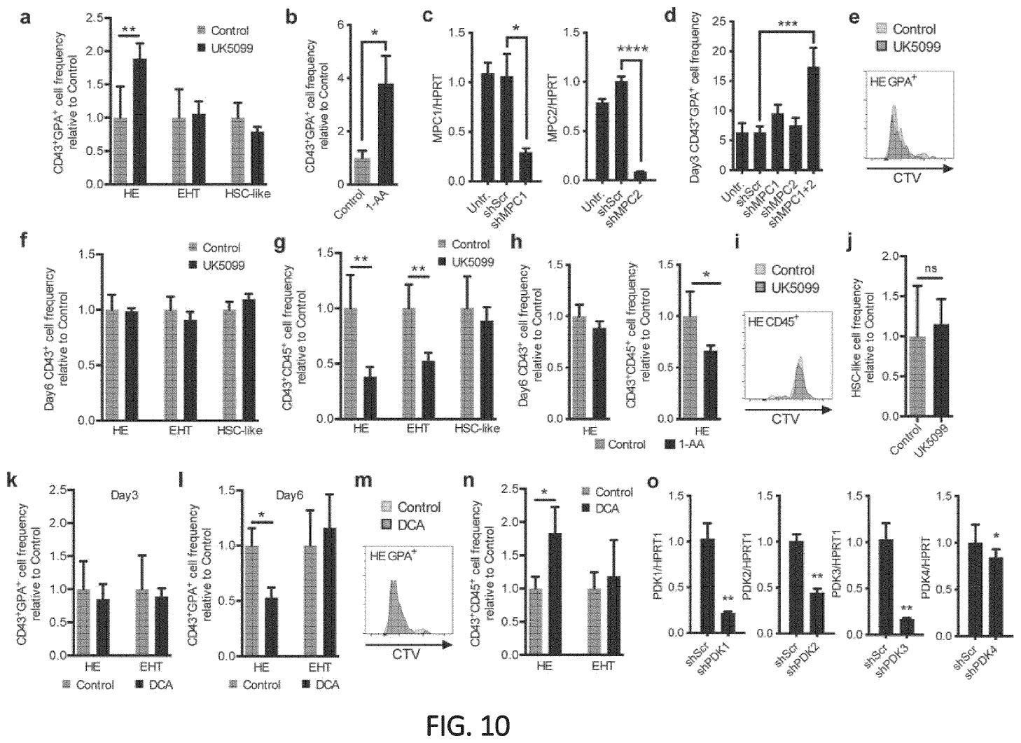

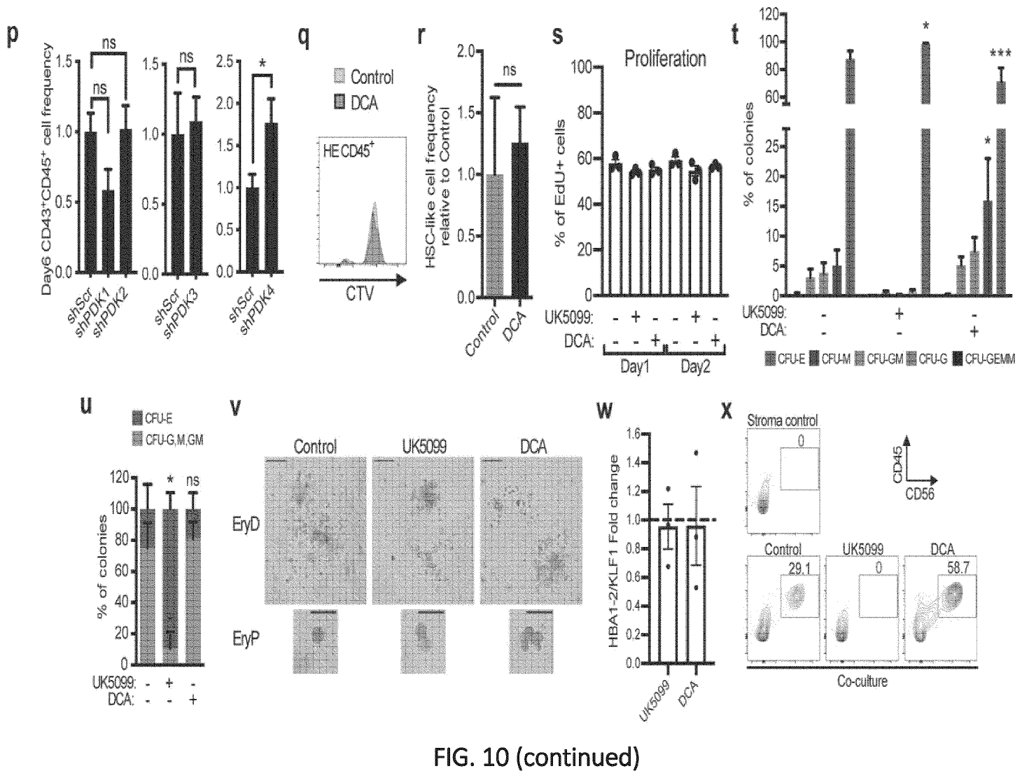

As we have shown that HE cells take up glucose at similar levels as EHT cells (FIG. 2, f) even though their glycolytic rates are lower, we investigated whether pyruvate oxidation is important for the hematopoietic differentiation of HE cells. Pyruvate is taken up by mitochondria via the mitochondrial pyruvate carrier complex (MPC) and can be converted to acetyl-CoA by the PDH enzyme to replenish the TCA cycle (FIG. 3, a). We blocked pyruvate entry into mitochondria using a specific MPC inhibitor called UK5099 (FIG. 3, a). In HE cells, unlike in EHT or HSC-like cells, MPC inhibition led to a striking increase in CD43‘GPA’ cell output at day 3 of subculture (FIG. 3, b and FIG. 10, a). To confirm this result, we treated HE cells with an alternative molecule that also blocks pyruvate use in the TCA cycle, a PDH inhibitor (Takubo, K. et al. Regulation of Glycolysis by Pdk Functions as a Metabolic Checkpoint for Cell Cycle Quiescence in Hematopoietic Stem Cells. Cell Stem Cell 12, 49-61 (2013)) (FIG. 3, a) and observed a significant increase in GPA⁺ cell output compared to the control (FIG. 10, b). Furthermore, we downregulated the expression of both MPC subunits, MPC1 and MPC2, using lentiviral vectors expressing shRNAs (FIG. 10, c) and observed once more a 2.7-fold increase in CD43⁺GPA⁺ cell output at day 3 of subculture (FIG. 10, d), confirming our results with UK5099. We did not observe any differences in the proliferation of the GPA⁺ population deriving from HE in the presence of UK5099 as compared to the control (FIG. 10, e). These results demonstrate that the use of glucose for glycolysis is sufficient to drive erythroid cell formation and that inhibiting pyruvate entry into mitochondria leads to an increased differentiation of HE cells toward the erythroid lineage. Furthermore, this biasing occurs on HE cells prior to full hematopoietic cell commitment, as demonstrated by the absence of bias when using cells downstream of the HE such as the EHT and HSC-like populations (FIG. 10, a).

Although the levels of total CD43⁺ cells were unchanged between UK5099-treated and untreated conditions at day 6 (FIG. 10, f), we observed a 2-fold decrease in the CD43⁺CD45⁺ populations deriving from both HE and EHT cells, but not from HSC-like cells (FIG. 3, c, FIG. 10, g). Similarly, 1-AA treatment led to a significant decrease in HE-derived CD45⁺ cell population, even though total CD43⁺ cell levels were unchanged (FIG. 10, h). It is important to note that UK5099 did not have an effect on the proliferation of CD45⁺ cells or on the frequency of HSC-like cells, both deriving from HE (FIG. 10, i, j). These results indicate that blocking pyruvate entry into mitochondria impairs differentiation towards a CD45⁺ hematopoietic fate during EHT.

To complement these findings, we sought to induce an opposite effect by increasing pyruvate flux into mitochondria to promote TCA cycle activity and OXPHOS. Using DCA, we blocked PDKs which repress the PDH complex: this allows pyruvate to be converted to acetyl-CoA and potentially fuel the TCA cycle (FIG. 3, a). Although the formation of CD43⁺GPA⁺ cells was not significantly altered by DCA at day 3 of HE subculture (FIG. 10, k), we observed a 50% decrease in this population at day 6 in the treated condition (FIG. 3, d, FIG. 10, l). Thus, as DCA does not directly block glycolysis, it does not affect primitive erythroid differentiation from HE cells. Indeed, the proliferation of the GPA⁺ population deriving from the HE subculture was not affected by DCA at day 3 of subculture (FIG. 10, m). Importantly, at day 6 of subculture with DCA, we observed an 80% increase in the percentage of CD43⁺CD45⁺ cells deriving from HE, but not EHT cells (FIG. 3, e; FIG. 10, n). Moreover, to validate this result, we downregulated the expression of PDKs using shRNAs (FIG. 10, o) and while we did not note a significant effect of PDK1, PDK2 or PDK3 downregulation, we observed a 75% increase in CD43⁺CD45⁺ cell output at day 6 of subculture following PDK4 downregulation (FIG. 10, p). This confirms our results with DCA and furthermore shows that its effect is related to blocking PDK4 specifically. The proliferation of CD45⁺ cells or the frequency of HSC-like cells deriving from HE was not affected by DCA (FIG. 10, q, r). To assess if the changes in lineage biasing seen with the metabolic regulators was due to changes in proliferation, we also performed EdU incorporation at early time points of subculture (days 1 and 2). We found no differences in proliferation due to UK5099 or DCA treatments (FIG. 10, s), further suggesting that the changes in lineage outcomes are the result of changes in the lineage potential in the HE. Taken together, these results show that when glycolysis is promoted via inhibition of pyruvate entry into the TCA cycle, HE cells preferentially give rise to erythroid cells; conversely when pyruvate is pushed towards oxidation in mitochondria, the increased TCA cycle fueling favors CD45+ differentiation of HE cells.

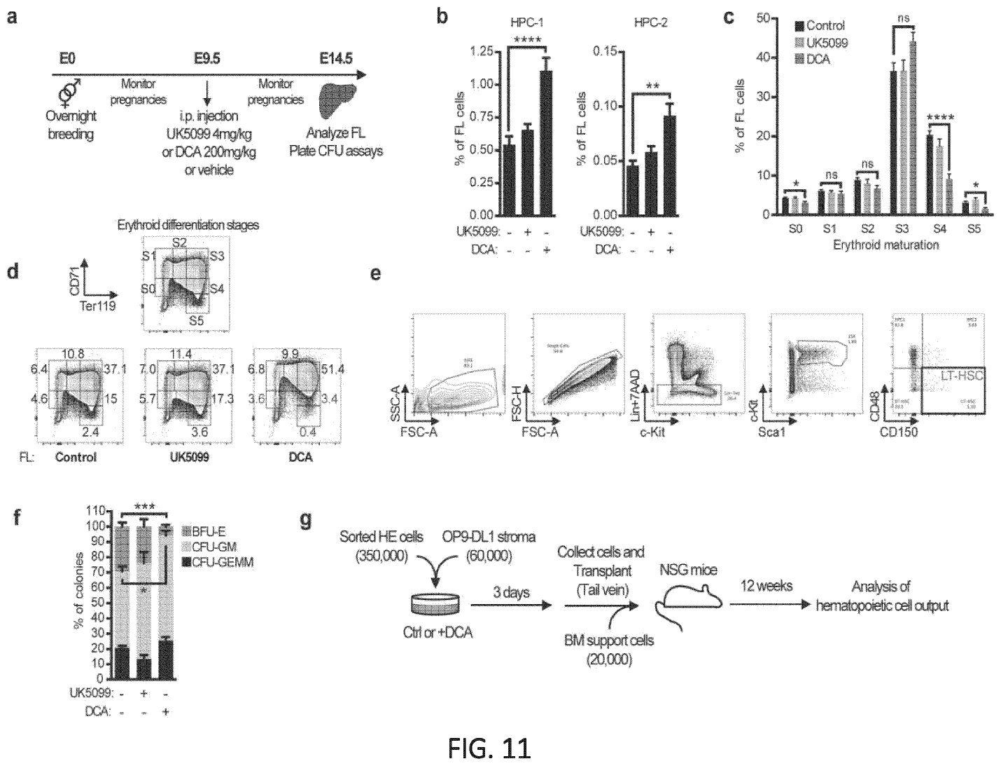

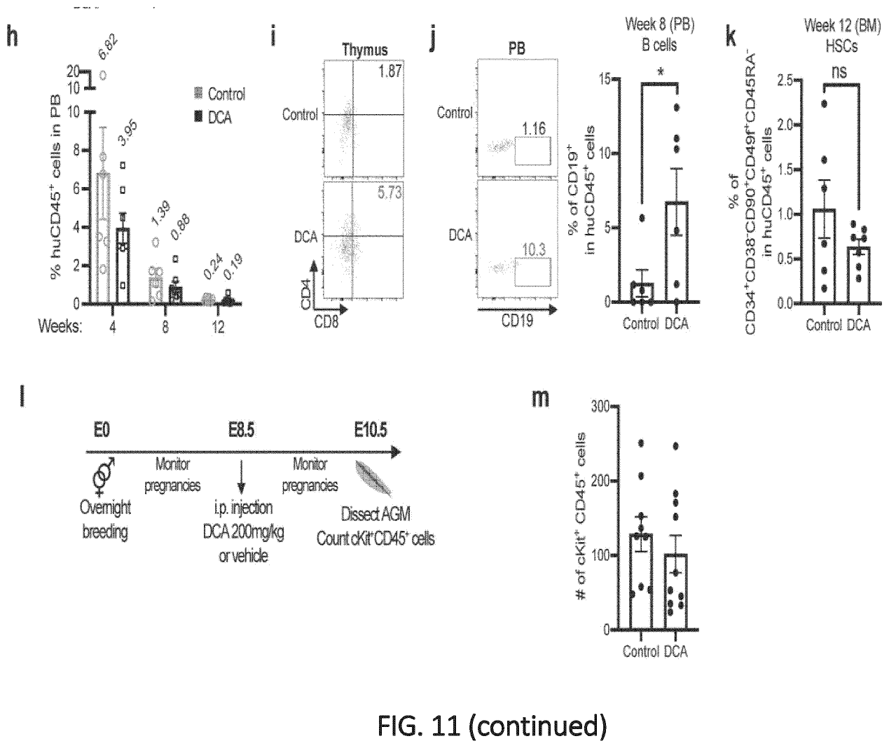

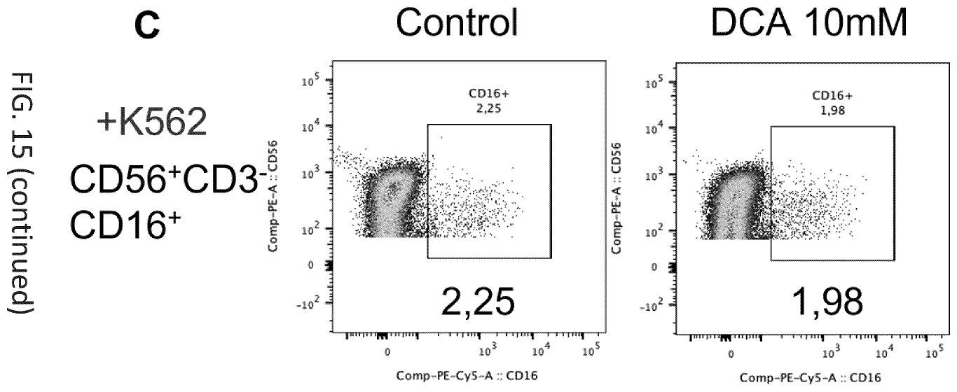

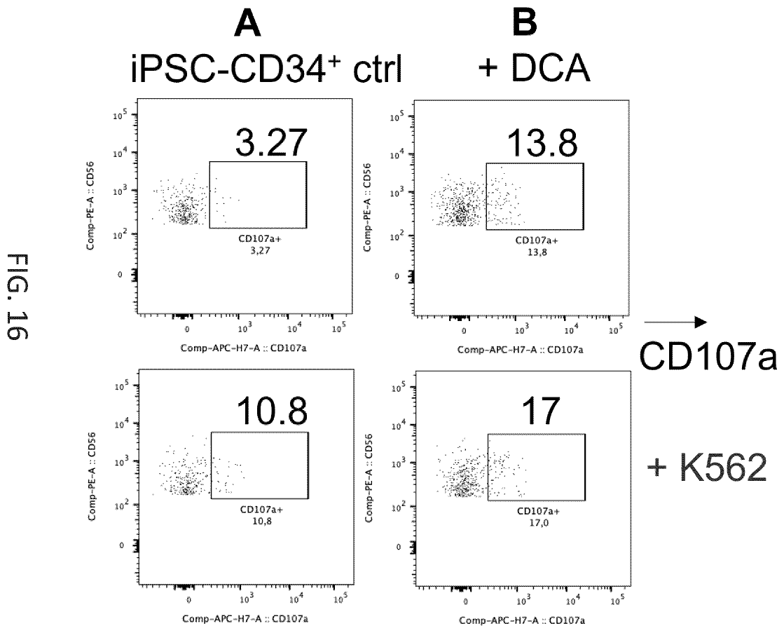

Following 3 or 6 days of MPC inhibition with UK5099 in HE cells, we found that erythroid colony (CFU-E) formation was significantly increased compared to the untreated condition, while granulocyte and macrophage colonies were decreased (CFU-G, GM and M) (FIG. 10, t, u). In contrast, while a 3-day PDK inhibition with DCA did not have an effect on CFUs (FIG. 10, u), a 6-day DCA treatment led to a decrease in CFU-Es and a significant increase in CFU-M colonies (FIG. 10, t). The ratio of CFU-E colonies to the sum of CFU-G, CFU-GM and CFU-M colonies was 20-fold higher in UK5099-treated cells and more than 3-fold lower in DCA-treated cells, as compared to the control (FIG. 3, f). In all conditions, we observed both bright red primitive (EryP) and brownish definitive erythroid (EryD) colonies (FIG. 10, v). However, while UK5099 or DCA did not have an effect on HBA1-2 (adult globin) expression (FIG. 10, w), we observed a significant increase in HBE1 (embryonic) and HBG1-2 (fetal) globin transcripts in colonies obtained from UK5099-treated HE cells (FIG. 3, g), confirming that MPC inhibition increases generation of erythroid cells with globin gene expression associated with primitive hematopoiesis. On the other hand, to understand whether DCA induces the formation of definitive hematopoietic cells, we induced lymphoid differentiation of day 3 HE cells in OP9-DL1 stroma co-cultures. While UK5099 treatment impaired NK cell formation, DCA (3 mM) treatment significantly increased NK cell differentiation as compared to untreated HE cells (FIG. 3, h; FIG. 10, x). The increased NK cell differentiation was also observed with DCA treatment at a higher dose of 10 mM (FIG. 15A), which also resulted in increased NK cell activation (FIG. 15B). Furthermore, treatment with DCA at 10 mM did not impact the activity of the NK cells, which were similarly depleted following the co-culturing with an exemplary target cancer cell, K562 (FIG. 15C), which demonstrates that the NK cells derived from source cells treated with a metabolic regulator retain anti-tumour activities. Altogether, these results demonstrate that HE cells can be directed towards differing hematopoietic lineage outcomes whereby promoting pyruvate use in glycolysis (using UK5099) increases erythropoiesis with embryonic and fetal globin expression, and conversely promoting pyruvate use for OXPHOS (using DCA) increases lymphoid lineage output. FIG. 3, h shows a 3-fold increase in NK cells output with the OXPHOS promoting regulators. Upon transplantation of our iPS derived cells into mice, there is a high-level engraftment with lymphoid progenitors (FIG. 11, g and h). FIG. 3, n shows a 3-fold increase of CLP (common lymphoid progenitor).

Thyroid hormone increases hepatic HMG-CoA reductase levels by acting to increase both transcription and stability of the mRNA. HMG-CoA reductase is a molecule that catalyzes a rate limiting step in cholesterol biosynthesis. Thyroid hormone acts on liver cells in the body. It is well known that some blood cells have the thyroid hormone receptor, which is well known to be involved in red blood cell production. Therefore, the thyroid hormone receptor is likely to be present on other blood cells and the hemogenic endothelium.

To verify these findings in an in vivo setting, we injected pregnant mice with UK5099 or DCA at embryonic day (E) 9.5 (FIG. 11, a), to influence hemogenic endothelium which gives rise to definitive hematopoiesis (both the second and third waves) occurring at E9-9.5 and E105, but not primitive hematopoiesis which takes place at E7-7.25 (1; and 4). We assessed blood lineage output in embryos by characterizing the cellular composition of fetal liver (FL) at E14.5 when the FL is the prime site of hematopoiesis. We observed that hematopoietic progenitor cells (HPC)-1, which are restricted progenitors with lymphoid/myeloid potential and HPC-2, which mainly give rise to megakaryocytic progeny were significantly increased in embryos from DCA-injected mice, as compared to the control and UK5099-injected conditions (FIG. 11, b). In line with this, both T and B cell levels were increased in DCA versus control and UK5099-injected embryos (FIG. 3, i), supporting our in vitro results showing an increased CD45⁺ output with DCA. Moreover, DCA treatment led to significant decreases in stage 0, 4 and 5 erythroid populations in the FL, with no significant differences in stages 1, 2 and 3 as compared to the control and UK5099 conditions (FIG. 11, c and d). This profile suggests an impairment in definitive erythroid cell production (decrease in S0), while primitive erythrocytes that have formed prior to the injection are in late maturation stages in the FL (SI, 2 and 3) or have exited from the FL into the circulation (decrease in S4 and 5), as described previously (Fraser, S. T., Isern, J. & Baron, M. H. Maturation and enucleation of primitive erythroblasts during mouse embryogenesis is accompanied by changes in cell-surface antigen expression. Blood 109, 343-352 (2007); and Isern, J., Fraser, S. T., He, Z. & Baron, M. H. The fetal liver is a niche for maturation of primitive erythroid cells. PNAS 105, 6662-6667 (2008)).

Furthermore, while the frequency of phenotypic long-term HSCs (LT-HSCs) defined as LSK CD48⁻CD150⁺ (FIG. 11, e) from the treated embryos were not significantly affected by UK5099 or DCA (FIG. 3, j), confirming our in vitro findings (FIG. 10, j and r), LT-HSCs from DCA16 treated embryos did give rise to significantly more CFU-GM colonies and fewer BFU-E colonies (FIG. 11, f) with an 80% decrease in the BFU-E to CFU-GM ratio (FIG. 3, k), as compared to the control and UK5099-treated conditions. We did not see a significant effect on in vivo EHT and hematopoiesis by UK5099 (FIG. 3, i-k), confirming that MPC inhibition preferably affects the primitive hematopoietic wave. Thus, analogous to our results in vitro, promoting pyruvate entry to the TCA cycle by DCA increases the frequency of lymphoid/myeloid cells at the expense of mature erythroid cells in vivo.

In order to assess definitive hematopoietic potential of iPSC-derived cells, we intravenously injected 3-day DCA-treated HE cells co-cultured with OP9-DL1 stroma into irradiated NSG mice (FIG. 11, g). We obtained engraftment levels comparable to previous studies (Rahman, N. et al. Engineering the haemogenic niche mitigates endogenous inhibitory signals and controls pluripotent stem cell-derived blood emergence. Nat Commun 8, 1-12 (2017)), with around 1% human CD45⁺ cells in the peripheral blood (PB) at week 8 (FIG. 11, h). Intriguingly, at 12 weeks, we detected significantly more CD4⁺CD8⁺ DP thymocytes in the thymi of NSG mice injected with DCA-treated HE cells (FIG. 3, l; FIG. 11, i). Furthermore, with DCA-treated cells we detected significantly more human B cells in the PB and BM of the NSG mice at weeks 8 and 12, respectively (FIG. 3, m and FIG. 11, j). In agreement with this, analysis of week 12 BM revealed a significant increase in the common lymphoid progenitor (CLP) population in the DCA treated condition (FIG. 3, n) despite similar levels of phenotypic human HSCs (defined as CD34⁺CD38⁻CD90⁺CD49f⁺CD45RA⁻) in both conditions (FIG. 11, k). In contrast, the frequencies of the CD11b⁺ myeloid lineage cell were similar between the conditions (FIG. 3, o, p). Taken together, these transplantation results show that promoting OXPHOS by increasing pyruvate flux into mitochondria with DCA pushes HE cells towards a definitive hematopoietic phenotype with T and B potential.

Since we did not see a difference in the frequency of HSC-like cells in vitro (FIG. 10, r) LT-HSCs in vivo (FIG. 3, j) or human HSCs deriving from transplanted HE cells in vivo (FIG. 11, k) following DCA treatment, we next sought to understand whether DCA could have an effect directly on the EHT process by affecting the numbers of resulting hematopoietic stem and progenitor cells (HSPCs). As this process takes place at E10.5 in the AGM region (Medvinsky, A. & Dzierzak, E. Definitive Hematopoiesis Is Autonomously Initiated by the AGM Region. Cell 86, 897-906 (1996)), we treated pregnant mice with DCA at E8.5 and assessed HSPC cell numbers (cKit⁺CD45⁺ cells) in the AGM regions of embryos at E10.5 (FIG. 11, l). We found that the numbers of cKit⁺CD45⁺ cells in control and DCA treated embryos were similar and in the same range as previously described (Morgan, K., Kharas, M., Dzierzak, E. & Gilliland, D. G. Isolation of Early Hematopoietic Stem Cells from Murine Yolk Sac and AGM. J Vis Exp (2008) doi:10.3791/789) (FIG. 11, m). This result confirms our in vitro and in vivo results and suggests that the modulation of pyruvate metabolism has an intrinsic effect in HE cells which is inherited by HSPCs without affecting their numbers.

### Example 5—Pyruvate Fate Dictates Hematopoietic Lineage Specification of HE Cells at the Single-Cell Level

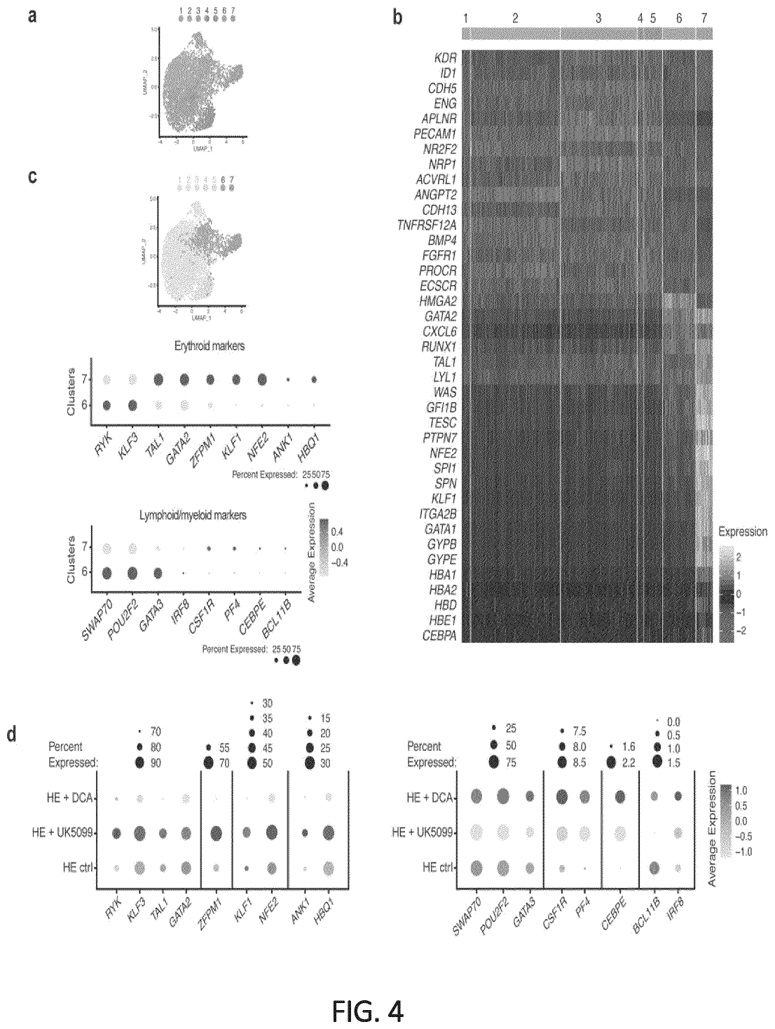

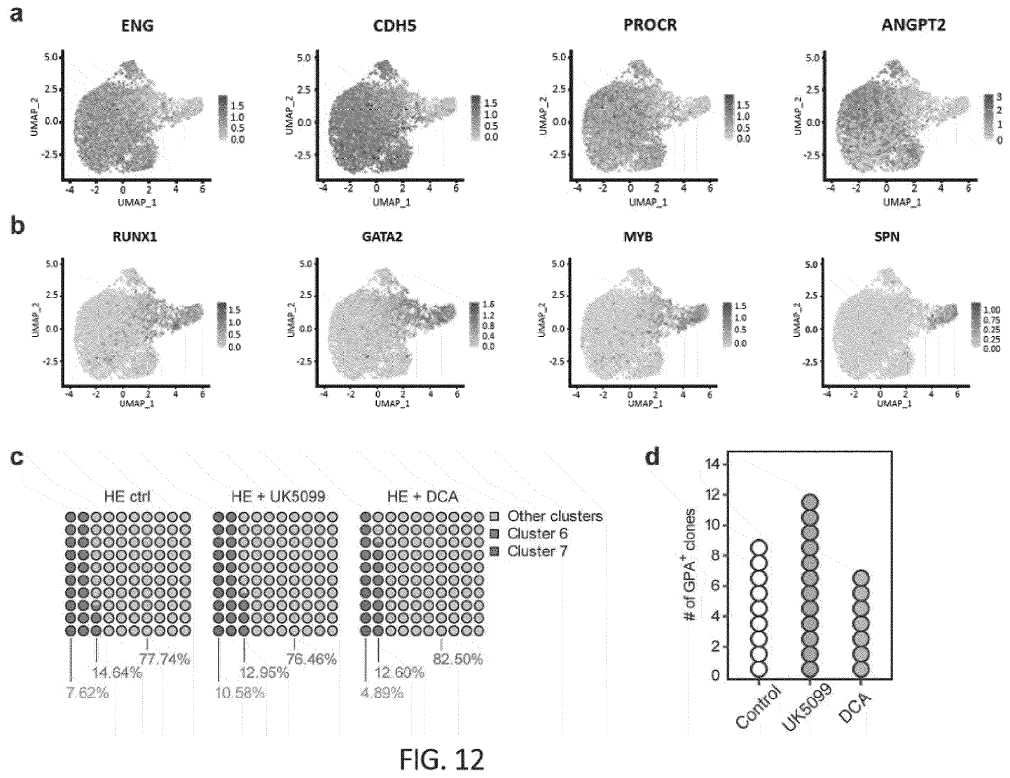

In order to dissect the molecular effects of pyruvate manipulation on HE cells, we assessed the transcriptomic profiles of these cells at an early time point of treatment (day 2), at the single cell level, in control and UK5099- or DCA-treated cells. First, we grouped all conditions together and separated the cells into 7 clusters (FIG. 4, a). The majority of HE cells expressed endothelial markers including ENG, CDH5, PROCR and ANGPT2 (FIG. 12, a) and their expression was mostly confined to clusters 1 through 5 (FIG. 4, b). In contrast, cells in clusters 6 and 7 expressed hematopoietic genes including RUNX1, GATA2, MYB and SPN (FIG. 4, b and FIG. 12, b). Thus, this time point most likely captures the commitment of HE cells to hematopoietic cells which occurs within clusters 6 and 7.

Focusing on isolated clusters 6 and 7 (FIG. 4, c), we found that an early erythropoiesis regulator, RYK, and erythroid-specific KLF3, were already expressed in cluster 6 at high levels, while several other members of the erythroid transcription factor network such as TAL1, GATA2, ZFPM1, KLF1, NFE2, ANK1 and HBQ1 were more highly expressed in cluster 7 (Erythroid markers; FIG. 4, c). Early lymphoid cell fate regulators POU2F2 (B cell) and GATA3 (T cell) as well as myeloid markers SWAP70 and IRF8 were expressed at higher levels in cluster 6, while T lymphoid BCL11B, myelo-monocytic CSF1R, CEBPE, and megakaryocytic PF4 were highest in cluster 7 (Lymphoid/myeloid markers; FIG. 4, c). Thus, while cluster 6 cells expressed early regulators of specific lineages, cluster 7 cells started expressing transcription factors, which are part of lineage-specific transcription factor networks characteristic of more mature hematopoietic cells. Moreover, in cluster 7, the percentage of cells expressing erythroid transcription factors was more than 75%, while cells expressing lymphoid or myeloid markers represented less than 20% of total (FIG. 4, c), in accordance with the early and late emergence of GPA⁺ and CD45⁺ cells, respectively, from HE.

We found that while the percentage of cells in cluster 6 was constant between conditions, there were 38% more UK5099-treated HE cells and 35% less DCA-treated HE cells in cluster 7 compared to the control (FIG. 12, c). This result shows that pyruvate modulation does not affect early hematopoietic commitment (cluster 6). However, it seems to have an effect on lineage specification (cluster 7).

We then confirmed that, in clusters 6 and 7, the average expression levels of erythroid lineage genes RYK, KLF3, TAL1, GATA2, ZFPM1, KLF1, NFE2, ANK1 and HBQ1 were higher in UK5099-treated HE cells as compared to the untreated HE cells and these factors were nearly absent in DCA-treated HE cells (left-hand dot plot, FIG. 4, d). In contrast, DCA-treated HE cells expressed higher levels of lymphoid/myeloid transcription factors SWAP70, POU2F2, GATA3, CSF1R, PF4, BCL11B, CEBPE and IRF8 compared to control and UK5099-treated HE cells (right-hand dot plot, FIG. 4, d). To further assess the effect of pyruvate manipulation on a single cell level, we sorted single HE cells onto OP9-DL1 stroma and scored GPA⁺ clones at day 14. From a total of 552 single cells per condition, we detected 12 GPA⁺ clones in the UK5099-treated condition and 7 GPA⁺ clones in the DCA-treated condition as compared to 9 GPA⁺ clones in the control (FIG. 12, d). This result highly suggests the preferential differentiation of HE cells to the erythroid lineage in the presence of UK5099. Taken together, these results show that at early stages of HE differentiation, modulation of pyruvate use directly impacts the expression of lineage-specific transcription factors and affects the resulting lineage specification of HE cells.

### Example 6—Primitive Erythroid Biasing of HE During MPC Inhibition Relies on LSD1

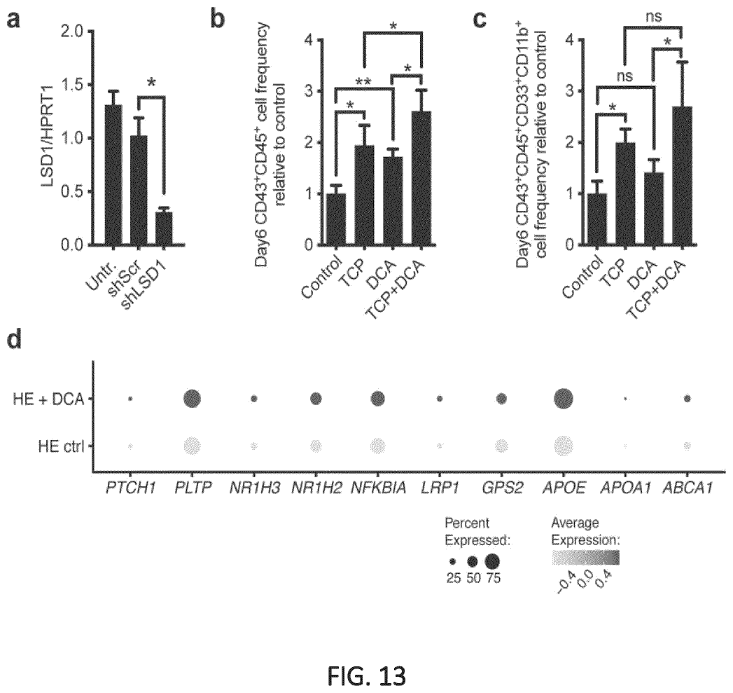

Previous studies have shown that Lysine-Specific Demethylase 1 (LSD1) is essential for EHT and particularly the erythroid lineage (Takeuchi, M. et al. LSD1/KDM1A promotes hematopoietic commitment of hemangioblasts through downregulation of Etv2. PNAS 112, 13922-13927 (2015); and Thambyrajah, R. et al. GFI1 proteins orchestrate the emergence of haematopoietic stem cells through recruitment of LSD1. Nat Cell Biol 18, 21-32(2016)). During EHT, LSD1 acts in concert with HDAC1/2 (Thambyrajah, R. et al. HDAC1 and HDAC2 Modulate TGF-β Signaling during Endothelial-to-Hematopoietic Transition. Stem Cell Reports 10, 1369-1383 (2018)) and GFI1/GFI1B (Thambyrajah, R. et al. GFI1 proteins orchestrate the emergence of haematopoietic stem cells through recruitment of LSD1. Nat Cell Biol 18, 21-32 (2016)) to induce epigenetic changes. We confirmed that HDACs are essential for EHT using an HDAC1/2 inhibitor (Trichostatin A, TSA) which impaired the emergence of CD43⁺ hematopoietic cells, as CD43 levels only reached an intermediate level, suggesting a block during EHT (FIG. 5, a). Moreover, we observed that LSD1, GFI1 and GFI1B are expressed at higher levels in UK5099-treated cells as compared to DCA-treated cells (FIG. 5, b), suggesting that the erythroid differentiation bias by pyruvate catabolism modulation may be LSD1-dependent. Strikingly, under conditions where LSD1 was blocked with Tranylcypromine (TCP), or downregulated by shRNAs expressed within a lentiviral vector (FIG. 13, a), we could not detect an increase in CD43⁺GPA⁺ cell frequency at day 3 following UK5099 treatment of HE cells (FIG. 5, c, d). On the other hand, TCP-treated HE cells gave rise to more CD43⁺CD45⁺ cells at day 6 similarly to DCA treatment (FIG. 13, b); however, we determined that unlike DCA, TCP specifically increased myeloid differentiation (FIG. 13, c), as previously described in the literature (Schenk, T. et al. Inhibition of the LSD1 (KDM1A) demethylase reactivates the all-trans retinoic acid differentiation pathway in acute myeloid leukemia. Nat Med 18, 605-611 (2012)). Thus, mechanistically, the induction of primitive erythropoiesis through MPC inhibition is dependent on epigenetic regulation by LSD1 in HE cells.

### Example 7—DCA-Dependent Definitive Hematopoiesis is Promoted by Cholesterol Metabolism

Dichloroacetate may be directly used as a precursor of acetylation marks: acetate is converted to acetyl-CoA by ACSS2 and transferred onto histones via histone acetyltransferases (HATs) (FIG. 5, e). Inhibiting ACSS2 did not perturb the DCA effect on CD43+CD45+ cells at day 6 of HE subculture (FIG. 5, f), showing that DCA is not directly converted to acetyl-CoA. Moreover, blocking HATs with C646 alone did not have an effect on HE cells; however, C646+DCA treatment boosted the increase in CD43+CD45+ cells 2-fold compared to DCA alone (FIG. 5, g). As blocking HATs did not inhibit the DCA effect, it was not surprising to find no changes in global acetylation of H3K9 or H4 with DCA (FIG. 5, h). Thus, inhibiting HATs together with enhancing PDH activity might promote acetyl-CoA availability for other metabolic processes, leading to the increase in CD45+ cells. Acetyl-CoA is a precursor for both lipid biosynthesis (via ACC) and for the mevalonate pathway (via HMGCR) which produces cholesterol (FIG. 5, i). Blocking ACC with CP-640186 (CP) had the same effect as DCA and combined treatment with both CP and DCA further increased the frequency of CD43+CD45+ cells at day 6 compared to DCA alone (FIG. 5, j). Thus, preventing lipid biosynthesis may increase acetyl-CoA availability for cholesterol production. Indeed, in DCA-treated HE cells, we detected an increase in cholesterol content both by imaging (FIG. 5, k) and flow cytometry (FIG. 5, l) and the latter experiment showed a dose-dependent effect of DCA on cholesterol content. Moreover, we observed higher levels of cholesterol efflux genes at day 2 in DCA-treated cells (FIG. 13, d), suggesting their upregulation due to the higher cholesterol content in these cells. Strikingly, treating HE cells with DCA in combination with atorvastatin (Ato) (inhibitor of the mevalonate pathway which is responsible for cholesterol biosynthesis) abrogated the effect of DCA (FIG. 5, m). Similarly, in zebrafish, cholesterol biosynthesis was shown to activate hematopoiesis via Notch signaling (Gu, Q. et al. AIBP-mediated cholesterol efflux instructs hematopoietic stem and progenitor cell fate. Science 363, 1085-1088 (2019)). Therefore, we assessed the expression of genes implicated in the Notch signaling pathway and observed that NOTCH1, JAG1 and HES1 levels were higher in DCA-treated cells compared to the control (FIG. 5, n). Moreover, we evaluated Notch1 expression during HE subculture and strikingly, we detected an increased level of Notch1 in DCA25 treated HE-derived CD43+CD45+ cells as compared to the control (FIG. 5, o). Taken together, our results show that DCA promotes cholesterol biosynthesis via Notch1, which favors definitive hematopoietic differentiation of HE cells (FIG. 5, p).

### Example 8—Comparison of the Impact of Different Metabolic Regulators on NK Cell Anti-Tumour Activity

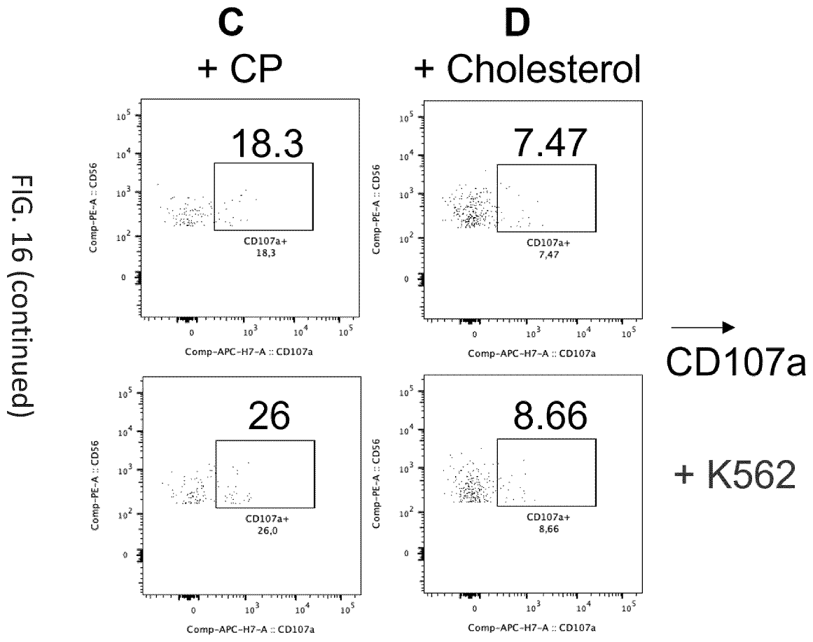

NK cell anti-tumour activity can occur via cytotoxic degranulation. CD107 is a marker that is present in the cytotoxic vesicles of NK cells, and so is not expressed on the cell surface of inactivated NK cells. However, upon NK cell activation and degranulation, the CD107 target becomes detectable on the cell surface. Therefore, the level of CD107 detected on NK cells correlates with cytotoxic degranulation. In this assay, several exemplary metabolic regulators were used to assess cytotoxic degranulation in a co-culture assay with K562 cells (an exemplary cancer cell model). As a negative control, iPSC-CD34⁺ untreated with a metabolic regulator were used, in which a basal level of 3.27% of degranulated NK cells (CD3⁻CD56⁺CD16⁺CD107⁺ cells) were observed (FIG. 16A, top panel). Interestingly, addition of DCA (10 mM) substantially increased degranulation to 13.8% of the population, i.e. more than 4-fold increase in degranulated NK cells (FIG. 16B, top panel); addition of CP (2.5 μM; Sigma) further increased degranulation to 18.3% of the population, i.e. more than 5-fold increase in degranulated NK cells (FIG. 16C, top panel); and cholesterol (as a cholesterol lipid concentrate, 0.5× concentration, Thermo Fisher 12531018 (250×)) also increased degranulation to 7.47% of the population, i.e. more than 2-fold increase in degranulated NK cells (FIG. 16D, top panel). Co-culturing with an exemplary cancer cell target (K562) also enhanced degranulation for all tested conditions, with the control iPSC-CD34⁺ untreated with a metabolic regulator increasing to 10.8% of the population (FIG. 16A, bottom panel). Furthermore, for all metabolic regulators used (DCA, CP and Cholesterol), the level of degranulated NK cells further increased when exposed to K562 cells, demonstrating that the treatment with various metabolic regulator types does not impact the enhancement of degranulation in the presence of cancer cells.

### Materials and Methods Related to Above-Described Examples

hiPSC Culture, Hematopoietic Differentiation and Cell Isolation

The RB9-CB1 human iPSC line (Woods, N.-B. et al. Brief report: efficient generation of hematopoietic precursors and progenitors from human pluripotent stem cell lines. Stem Cells 29, 1158-1164 (2011)) was co-cultured with mouse embryonic fibroblasts (MEFs, Millipore), passaged every six days and processed to form embryoid bodies (EBs) as described previously (Guibentif, C. et al. Single-Cell Analysis Identifies Distinct Stages of Human Endothelial to-Hematopoietic Transition. Cell Reports 19, 10-19 (2017)). The differentiation protocol used in this study was previously described (Ditadi, A. & Sturgeon, C. M. Directed differentiation of definitive hemogenic endothelium and hematopoietic progenitors from human pluripotent stem cells. Methods 101, 65-72 (2016)), however, small modifications were made to induce both primitive and definitive hematopoiesis, as indicated below and in FIG. 6, a. Newly-formed EBs were first kept in SFD medium supplemented with 1 ng/ml Activin A (on days 0-2) and 3 μM CHIR99021 (on day 2 only). At day 3, media was switched to “Day 3-SP34” medium supplemented with 1 ng/ml Activin A (on day 3 only) and 3 μM CHIR99021 (on day 3 only) until day 6. On day 6, media was replaced by “Day 6-SP34” medium, until day 8. In some experiments where indicated, to obtain a higher yield of HSC-like cells, EBs were kept until day 10: in this case, EBs were plated onto Matrigel (8 μg/cm2, Corning)-coated dishes on day 8 and kept until day 10. Media was changed every day, except on days 5 and 7. On day 8 or 10 (as indicated), EBs were singularized with 5-6 rounds of 5-minute incubations with TryPLE Express (Thermo Fisher Scientific). CD34⁺ cells were selected using the human CD34 MicroBead kit (Miltenyi Biotec) and stained with CD34-FITC, CD73-PE, VECad-PerCPCy5.5, CD38-PC7, CD184-APC, CD45-AF700, CD43-APCH7, GPA-eF450, CD90-BV605 and the viability marker 7AAD in order to sort HE (CD34⁺CD43⁻CXCR4⁻CD73⁻CD90⁺VECad⁺), EHT (CD34⁺CD43^{int}CXCR4⁻CD73⁻CD90⁺VECad⁺) and HSC-like (CD34⁺CD43⁺CD90⁺CD38⁻) cells, according to previously described markers (Guibentif, C. et al. Single-Cell Analysis Identifies Distinct Stages of Human Endothelial to-Hematopoietic Transition. Cell Reports 19, 10-19 (2017); Ditadi, A. et al. Human definitive haemogenic endothelium and arterial vascular endothelium represent distinct lineages. Nat Cell Biol 17, 580-591 (2015); and Choi, K.-D. et al. Identification of the Hemogenic Endothelial Progenitor and Its Direct Precursor in Human Pluripotent Stem Cell Differentiation Cultures. Cell Reports 2, 553-567 (2012)).

### HE, EHT and HSC-Like Subculture

Sorted HE (40,000), EHT (30,000) and HSC-like (5-20,000) cells were plated onto Matrigel (16 μg/cm2, Corning)-coated 96-well flat bottom plates in HE medium with 1% penicillin-streptomycin and kept in a humidified incubator at 37° C., 5% C02, 4% 02 overnight. The following day (day 0), wells were washed twice with PBS and fresh HE medium was added, together with 2-DG (1 mM), UK5099 (10 μM), DCA (3 mM or 10 mM), TSA (60 nM), TCP (300 nM), ACSS2i (5 μM), C646 (10 μM), CP-640186 (5 μM) or Atorvastatin (0.5 μM), where indicated. Media was changed and drugs were added every 2 days and cells were kept in a humidified incubator at 37° C., 5% CO₂, 20% O₂ for 6-7 days. Pictures were taken using an Olympus IX70 microscope equipped with a CellSens DP72 camera and CellSens Standard 1.6 software (Olympus). For single cell subcultures, single HE cells were directly sorted with a BD FACSAriaIII onto OP9-DL1 stroma in flat-bottom 96-well plates with 120 μl OP9 medium (OptiMEM medium with Glutamax (Invitrogen) with 10% FCS, 1% penicillin-streptomycin solution (Thermo Fisher Scientific) and 1% 2-mercaptoethanol (Invitrogen)) with SCF, IL-6, IL-11, IGF1 and EPO, and with or without 10 μM UK, or 3 mM or 10 mM DCA. The cells were kept in a humidified incubator at 37° C., 5% CO₂, 4% O₂ and media was replaced every 4 days. At day 14, the wells were collected and scored for GPA+ clones by flow cytometry, using a BD LSRII.

### Extracellular Flux Analyses

For comparisons between HE, EHT and HSC-like cells, day 10 FACS-sorted cells (≥40,000) were directly plated onto Seahorse XF96 Cell Culture Microplate wells coated with CellTak (0.56 μg/well) in 2-4 replicates and extracellular flux was assessed immediately on a Seahorse XF96 analyzer. For comparisons between HE and EHT cells, day 8 FACS-sorted cells (≥40,000) were plated onto Matrigel (16 μg/cm², Corning)-coated Seahorse XF96 Cell Culture Microplate wells in 3-4 replicates and extracellular flux was assessed 2 days after plating, on a Seahorse XF96 analyzer. To assess glycolytic flux, ECAR was measured in XF medium with 2 mM glutamine under basal conditions (after 1-hour glucose starvation as per manufacturer's instructions) as well as after the addition of 25 mM glucose, 4 μM oligomycin and 50 mM 2-DG and data was normalized to cell number. The levels of glycolytic capacity (ECAR_{oligomycin}−ECAR_{2-DG}) and glycolysis (ECAR_{glucose}−ECAR_{2-DG}) were calculated. To assess oxidative phosphorylation, OCR was measured in XF medium with 10 mM glucose, 2 mM glutamine and 1 mM sodium pyruvate under basal conditions as well as after the addition of 4 μM oligomycin, 2 μM Carbonyl cyanide 4-(trifluoromethoxy)phenylhydrazone (FCCP) and 1 μM rotenone/40 μM Antimycin A and data was normalized to cell number. The levels of basal respiration (OCRbasal−OCR_{Rotenone/AntimycinA}), ATP-production (OCR_{basal}−OCR_{Oligomycin}) and maximal respiration (OCR_{FCCP}−OCR_{Rotenone/AntimycinA}) were calculated.

### Flow Cytometry Analyses

On days 3 and 6 of subculture, cells were collected after a 2-minute incubation at 37° C. with StemPro Accutase Cell Dissociation Reagent and stained with CD34-FITC, CD14-PE, CD33-PC7, CD11b-APC, CD45-AF700, CD43-APCH7, GPA-eF450, CD90-BV605 and the viability marker 7AAD and fluorescence was measured on a BD LSRII. To measure mitochondrial activity, cells were incubated with Tetramethylrhodamine ethyl ester (TMRE, 20 nM) for 30 minutes at 37° C. Negative controls were incubated with 100 μM FCCP for 30 minutes at 37° C., prior to TMRE staining. Fluorescence was measured on a BD FACSARIA III and MFI levels—MFI FMO were calculated. To measure glucose uptake, cells were incubated with 2-(N-(7-Nitrobenz-2-oxa-1,3-diazol-4-yl)Amino)-2-Deoxyglucose (2-NBDG) for 30 minutes at 37° C. and fluorescence was measured on a BD FACSARIA III. To measure proliferation, cells were processed with the CellTrace Violet (CTV) kit according to manufacturer's instructions (10-minute incubation) and fluorescence was measured on a BD LSRFortessa. To measure EdU incorporation, HE cells were assessed on day 1 or 2 of subculture after 24 h EdU pulses, using Click-iT EdU Flow Cytometry Cell Proliferation Assay (Thermo Fisher Scientific, C10424), according to manufacturer's instructions. Flow cytometry outputs were analyzed on FlowJo Software, with initial gatings on SSC-A/FSC-A, FSC-H/FSC-A, SSC-H/SSC-A and 7-AAD to exclude doublets and dead cells in all experiments.

### Colony Forming Unit Assay

Subcultured HE cells were treated with StemPro Accutase Cell Dissociation Reagent for 2 minutes at 37° C. and dissociated cells were resuspended in 3 ml Methocult H4230 (STEMCELL Technologies, France) (prepared according to manufacturer's instructions, with 20 mL Iscove's Modified Dulbecco's Medium containing 2.5 μg hSCF, 5 μg GM-CSF, 2.5 μg IL-3 and 500 U EPO). Each mixture was divided onto 2 wells of a non-tissue culture treated 6-well plate. Following a 12-day incubation in a humidified incubator at 37° C., 5% CO₂, 20% O₂, colonies were morphologically distinguished and scored. For globin analysis, colonies in Methocult wells were harvested with PBS, washed thoroughly and frozen in RLT buffer with β-mercaptoethanol. Following RNA extraction and RT (Qiagen), gene expression was assessed with taqman probes by q-PCR. The taqman probes used for this assay are HBA1/2 (Hs00361191_g1), HBE1 (Hs00362216_m1), HBG2/1 (Hs00361131_g1) and KLF1 (Hs00610592_m1).

### Lymphoid Differentiation Assay on OP9-DL1 Stroma

Subculture day 3 HE cells cultured in the presence of UK5099 (10 μM) or DCA (3 mM or 10 mM), as indicated, were collected after a 2-minute incubation at 37° C. with StemPro Accutase Cell Dissociation Reagent and seeded onto 80% confluent OP9-DL1 stroma. Cells were cultured in OP9 medium (OptiMEM medium with Glutamax (Invitrogen) with 10% FCS, 1% penicillin-streptomycin solution (Thermo Fisher Scientific) and 1% 2-mercaptoethanol (Invitrogen)) with SCF (10 ng/ml), FLT3-L (10 ng/ml), IL-2 (5 ng/ml), IL-7 (5 ng/ml, first 15 days only) and IL-15 (10 ng/ml) with passaging onto new OP9-DL1 stroma every week, as described previously (Renoux, V. M. et al. Identification of a Human Natural Killer Cell Lineage-Restricted Progenitor in Fetal and Adult Tissues. Immunity 43, 394-407 (2015)). At day 35 of co-culture, cells were analyzed on a BD LSRFortessa.

### Single-Cell RNAseq Library Preparation and Sequencing

Sorted HE, EHT and HSC-like cells as well magnetically selected (Miltenyi Biotec) cord blood CD34⁺ cells were plated onto Matrigel (16 μg/cm², Corning)-coated 96-well flat bottom plates in HE medium (Ditadi, A. & Sturgeon, C. M. Directed differentiation of definitive hemogenic endothelium and hematopoietic progenitors from human pluripotent stem cells. Methods 101, 65-72 (2016)) with 1% penicillin-streptomycin and kept in a humidified incubator at 37° C., 5% CO₂, 4% O₂ overnight. The following day (day 0), wells were washed twice with PBS and fresh HE medium was added, together with UK5099 (10 μM) or DCA (3 mM), where indicated. On day 1 and 2 (as indicated), cells were washed twice with PBS 0.04% UltraPure BSA and collected after a 2-minute incubation at 37° C. with StemPro Accutase Cell Dissociation Reagent. Cells were spun down, resuspended in PBS 0.04% UltraPure BSA, counted (yield between 8,000-18,000 cells) and library preparation was conducted according to the Chromium Single Cell 3′ Reagent kit v3 instructions (10× Genomics). Sequencing was performed on a NOVASeq 6000 from Illumina with the run parameters (28-8-0-91) recommended by 10× Genomics with a final loading concentration of 300 pM of the pooled libraries. Human Umbilical Cord Blood samples were collected from Skine University Hospital (Lund and Malmo) and Helsingborg Hospital with informed consents according to guidelines approved by the regional ethical committee.

### Single-Cell RNAseq Analysis

The data was processed and analyzed using Seurat v3.1.0, where cells were allowed to have up to 20% mitochondrial reads prior to log-normalization and finding the top 500 variable genes using the “vst” method. Cell cycle scores were calculated and the data was scaled regressing on mitochondrial content and the difference of the S and G2M score. Principal components were calculated prior to calculating a UMAP. Pseudotime trajectories describing two developmental routes were identified in our EHT dataset using Slingshot (Street, K. et al. Slingshot: cell lineage and pseudotime inference for single-cell transcriptomics. BMC Genomics 19,477 (2018)) along which the cells were ordered. The cells were then binned along each trajectory where the cell type composition of each bin was calculated as percentages. Cord blood CD34+ cells were mapped to our data and labeled using scCoGAPS (Stein-O'Brien, G. L. et al. Decomposing Cell Identity for Transfer Learning across 23 Cellular Measurements, Platforms, Tissues, and Species. Cell Syst 8, 395-411.e8 (2019)). CS13 data from Zeng et al. (Zeng, Y. et al. Tracing the first hematopoietic stem cell generation in human embryo by single-cell RNA sequencing. Cell Res 1-14 (2019)) was read and processed to make a UMAP from which the cells they name as “AEC” and “Hem” were identified. These 99 cells were mapped to our data and labeled using SCMAP59. Carnegie Stage 13 data from Zeng et al. was mapped to our EHT dataset using scCoGAPS where 10 patterns were identified, named according to their respective weights (as shown in Table 1) and then projected using projectR. Table 1 shows how we have generated cells from iPS that have the same expression profiles as those published by Zeng et al. in the actual human embryo. This demonstrates that our in vitro protocol to generate developmentally relevant hematopoietic cells and precursors is excellent in that it recapitulates what happens in the embryo. The CS 13 dorsal aorta dataset from Zeng et al. was mapped onto our EHT dataset using scCoGAPS. The patterns resulting from this analysis were classified as the cell types described by Zeng et al.

Each cell was assigned to the group that achieved the highest weight. An overview showing the relationship between cell-types and patterns was done by forming a contingency table on which correspondence analysis was performed using the ca package for R Differentially expressed genes were found using the FindAllMarkers function. Cell numbers for day 1 samples are as follows: HE=1451, EHT=1523, HSC-like=732. Cell numbers for day 2 samples are as follows: HE ctrl=1195, HE+UK5099=718, HE+DCA=2309. All assessed endothelial and hematopoietic genes were previously used in several publications to validate the EHT process (Zhou, F. et al. Tracing haematopoietic stem cell formation at single-cell resolution. Nature 533, 487-492 (2016); Swiers, G. et al. Early dynamic fate changes in haemogenic endothelium characterized at the single-cell level. Nat Commun 4, 2924 (2013); Ng, E. S. et al. Differentiation of human embryonic stem cells to HOXA+ hemogenic vasculature that resembles the aorta-gonad-mesonephros. Nature Biotechnology 34, 1168-1179 (2016); and Guibentif, C. et al. Single-Cell Analysis Identifies Distinct Stages of Human Endothelial to-Hematopoietic Transition. Cell Reports 19, 10-19 (2017)). For gene expression analyses, gene sets for glycolysis, oxidative phosphorylation and cholesterol efflux were downloaded from The Molecular signatures Database (MSigDB).

Downregulation Via shRNAs

Short-hairpin sequences recognizing the genes of interest were cloned into GFP-expressing pRRL26 SFFV vectors, embedded in a microRNA context for minimal toxicity, as described previously (Fellmann, C. et al. An Optimized microRNA Backbone for Effective Single-Copy RNAi. Cell Reports 5, 1704-1713 (2013)). Each lentivirus batch was produced in two T175 flasks of HEK 293T cells by co-transfecting 22 μg of pMD2.G, 15 μg of pRSV-Rev, 30 μg of pMDLg/pRRE and 75 μg of the shRNA vector using 2.5 M CaCl2. Medium was changed 16 hours after transfection and viruses were harvested 48 hours after transfection, pelleted at 20,000×g for 2 hours at 4° C., resuspended in 100 μl DMEM, aliquoted and kept at −80° C. The downregulation efficiency of each shRNA was measured by assessing the corresponding gene expression by qPCR in sorted GFP⁺ cells, 3 days after lentiviral transduction of cord blood CD34⁺ HSPCs. The taqman probes used for this assay are MPC1 (Hs00211484_m1), MPC2 (Hs00967250_m1), PDK1 (Hs01561847_m1), PDK2 (Hs00176865_m1), PDK3 (Hs00178440_m1), PDK4 (Hs01037712_m1), LSD1/KDM1A (Hs01002741_m1) and HPRT1 (Hs02800695_m1). HE cells were transduced by direct addition of lentivirus particles into the culture medium on the day after the sort.

### In Vivo Compound Injections and Murine Hematopoiesis Assessment

For fetal liver analysis, pregnant female C57Bl/6xB6.SJL mice were injected intraperitoneally at E9.5 with UK5099 (4 mg/kg) or DCA (200 mg/kg) or PBS (control). Embryos were harvested at E14.5 and individually weighed and processed. Fetal livers were dissected and homogenised in 800 μL ice cold PBS supplemented with 2% fetal bovine serum (FBS) and FL cells were washed in PBS with 2%/FBS. For the differentiated lineage panel, cells were stained with B220 and CD19 (B-cell markers)-PE, CD3e-APC, Ter119-PeCy7 and CD71-FITC and analyzed on a BD FACSARIA III. For the HSC panel, samples were first treated with ammonium chloride solution (STEMCELL Technologies, France) to lyse red blood cells, washed twice in ice cold PBS with 2% FBS, stained with CD3e, B220, Ter119, Gr1 (Lineage)-PeCy5, c-Kit-Efluor780, Sca1-BV421, CD48-FITC, CD150-BV605 and 7-AAD (for dead cell exclusion) and analyzed on a BD FACSARIA III. Flow cytometry outputs were analyzed on FlowJo Software, with initial gatings on SSC-A/FSC-A and FSC-H/FSC-A to exclude doublets. For plating of CFU assays, 100 LT-HSCs were sorted (gating strategy shown in FIG. 11, e) and resuspended in 3.0 mL Methocult M3434 (STEMCELL Technologies, France). Each mixture was divided onto 2 wells of a non-tissue culture treated 6-well plate. Following a 14-day incubation in a humidified incubator at 37° C., 5% CO₂, 20% O₂, colonies were morphologically distinguished and scored. For AGM analysis, pregnant female C57BV6.SJL mice were injected intraperitoneally at E8.5 with DCA (200 mg/kg) or PBS (control). Embryos were harvested at E10.5 and their AGM regions were individually processed as described previously (Fang, J. S., Gritz, E. C., Marcelo, K. L. & Hirschi, K. K. Isolation of Murine Embryonic Hemogenic Endothelial Cells. JoVE (Journal of Visualized Experiments) e54150-e54150 (2016) doi:10.3791/54150). Briefly, AGM regions were dissected and digested with Collagenase II (Life Technologies) in PBS supplemented with 10% fetal bovine serum (FBS) and cells in single cell suspension were washed in PBS with 10% FBS. Cells were stained with anti-cKit and anti-CD45 antibodies and analyzed on a BD LSRFortessa. Experiments and animal care were performed in accordance with the Lund University Animal Ethical Committee.

### NSG Mice Transplantations

Sorted human HE cells (350,000) were mixed with OP9-DL1 stroma (60,000) and subcultured for 3 days with or without DCA (3 mM) on Matrigel (16 μg/cm², Corning)-coated 12-well plates in HE medium (32). Between 100,000-150,000 cells from control or DCA samples were transplanted into sub lethally irradiated (300 cGy) 8-week-old female NOD/Cg-Prkdc^{scid} Il2^{tm1Wj1}/SzJ mice (NSG, The Jackson Laboratory) together with 20,000 whole BM support cells from C57BV6.SJL mice (CD45.1⁺/CD45.2⁺, in house breeding). Cells were transplanted in single cell solution in 250 μL PBS with 2% FBS through intravenous tail vein injection. Drinking water of transplanted NSG mice was supplemented with ciprofloxacin (125 mg/L, HEXAL) for 3 weeks after transplantation to prevent infection. Mice were housed in a controlled environment with 12-hour light-dark cycles with chow and water provided ad libitim. Experiments and animal care were performed in accordance with the Lund University Animal Ethical Committee.

Peripheral Blood Analysis after NSG Mice Transplantations

Peripheral blood (PB) was collected from the tail vein into EDTA-coated microvette tubes (Sarstedt, Cat #20.1341.100). Peripheral blood was lysed for mature erythrocytes in ammonium chloride solution (STEMCELL technologies) for 10 minutes at room temperature, washed and stained for cell surface antibodies for 45 minutes at 4° C., washed and filtered prior to flow cytometry analysis on the FACS AriaIII (BD). Flow cytometry outputs were analyzed on FlowJo Software, with initial gatings on SSC Oburoglu et al. A/FSC-A and FSC-H/FSC-A for doublet exclusion, on DAPI for dead cell exclusion and on huCD45/muCD45.1 for murine cell exclusion.

Bone Marrow Analysis after NSG Mice Transplantations

Bone marrow was analyzed at the 12-week transplantation endpoint. Mice were euthanized by spinal dislocation followed by the dissection of both right and left femurs, tibias and iliac bones. Bone marrow was harvested through crushing with a pestle and mortar and cells were collected in 20 mL ice-cold PBS with 2% FBS, filtered and washed (350×g, 5 min). Bone marrow cells were lysed for red blood cells (ammonium chloride solution, STEMCELL technologies) for 10 minutes at room temperature, washed and stained for cell surface antibodies for 45 minutes at 4° C., washed and filtered prior to FACS analysis on the FACS AriaIII (BD). Flow cytometry outputs were analyzed on FlowJo Software, with initial gatings on SSC-A/FSC-A and FSC-H/FSC-A for doublet exclusion, on DAPI or 7AAD for dead cell exclusion and on huCD45/muCD45.1 for murine cell exclusion.

Thymus Analysis after NSG Mice Transplantations

Whole thymus was harvested at the 12-week transplantation endpoint. The thymocytes were mechanically dissociated from connective tissue in the thymus by pipetting up and down in PBS with 2% FBS, followed by filtration through a 50 μm sterile filter. Erythrocyte contamination was removed by lysing the sample in ammonium chloride solution (STEMCELL technologies) for 10 minutes at room temperature. Samples were washed and spun down after and the pellet of thymocytes was resuspended in FACS buffer and stained for cell surface antibodies for 45 minutes at 4° C., washed and filtered prior to FACS analysis on the FACS AriaIII (BD). Flow cytometry outputs were analyzed on FlowJo Software, with initial gatings on SSC-A/FSC-A and FSC-H/FSC-A for doublet exclusion, on DAPI for dead cell exclusion and on huCD45/muCD45.1 for murine cell exclusion.

### Confocal Microscopy Imaging and Quantification

For TMRE staining, on day 3 of subculture, half of the culture medium was removed and cells were stained with 20 nM TMRE (Thermo Fisher Scientific, T669) by direct addition into the culture medium of a 2× concentrated solution. After a 20-minute incubation at 37° C., wells were carefully washed with PBS and fresh HE medium was added. During acquisition, cells were kept in a humidified incubator at 37° C., 5% CO₂, 20% O₂. For immunocytochemistry, subculture day 2 HE cells (plated on coverslips) were washed twice in PBS, fixed with 4% PFA for 15 minutes at RT and washed three times with PBS. For filipin staining, fixed cells were incubated with 100 μg/ml filipin III (Sigma-Aldrich, F4767) for 1 hour, washed three times with PBS and rinsed with distilled water before mounting with PVA/DABCO. For H3K9 and H4 acetylation staining, fixed cells were permeabilized and blocked 1 hour at RT with PBS+0.25% Triton X-100+5% normal donkey serum (blocking solution) followed by incubation overnight at 4° C. with primary antibodies diluted in blocking solution. Cells were then washed 2×5 min with PBS+0.25% Triton X-100 (TPBS) and 5 min with blocking solution before incubation with secondary antibodies 2 hours at RT diluted in blocking solution. Cells were later washed 5 min with TPBS containing 1 μg/ml Hoechst and twice with PBS before being rinsed with distilled water and mounted with PVA:DABCO. Images were obtained with the 10× (TMRE) or 20× (Filipin and acetylation) objective of a Zeiss LSM 780 confocal microscope using the Zen software and a 1.5× zoom (TMRE) or 0.6× zoom (Filipin and acetylation). Acquisition settings were the same for all images of each experiment, taking the same number of stacks. Intensity quantification was performed using the Fiji software as follows. For TMRE, using the brightfield channel, ROIs were selected for 5 spindle shaped and 5 round cells (randomly chosen) and average intensity for each ROI was calculated in a summatory Z-stack of the TMRE channel. For filipin and acetylation, the summatory Z-stack for the filipin channel was obtained and average intensity calculated. A total of 2-3 independent experiments with 2-3 replicate wells were quantified. For each replicate well, 4-6 images were acquired.

### Statistical Analyses

Significance of differences between conditions were calculated using paired/unpaired t-tests, 1/2-way analysis of variance (ANOVA) tests or Kruskal-Wallis tests with multiple comparisons in GraphPad Prism 6 software, as indicated. p values are indicated in figures with the following abbreviations: ns, not significant, *p<0.05, **p<⁰0.01, ***p<0.001, ****p<0.0001.

### Data Availability

The single cell RNA sequencing data presented herein is available in the GEO database under the accession number GSE141189.

The present application also provides aspects according to the following paragraphs:

1. A method of generating a hematopoietic cell, comprising:
providing a source cell; and
treating the source cell with a metabolic regulator that directs the source cell to preferentially use glycolysis or oxidative phosphorylation
wherein the source cell is differentiated into a GPA⁺ erythroid cell or a CD45⁺ non-erythroid cell.

2. The method of paragraph 1, wherein the source cell is selected from the group consisting of a hemogenic endothelial (HE) cell, an iPS cell (such a differentiating iPS cell), a cell directly reprogrammed to a known pre-cursor of a hematopoietic cell, a cell directly reprogrammed to a hematopoietic cell or precursor of a hematopoietic cell, a reprogrammed cell that is subsequently further reprogrammed to a hematopoietic cell or precursor of a hematopoietic cell, an adult hematopoietic cell derived from bone marrow or mobilized peripheral blood and a neonatal hematopoietic cell derived from cord blood or prenatal tissue (e.g. placenta).

3. The method of paragraph 1, wherein the metabolic regulator is a molecule, a drug, protein, or RNA based system that regulates metabolic processes.

4. The method of paragraph 1, wherein the metabolic regulator is a viral vector, or an RNA-based system, or CRISPR/CAS-based system that regulates metabolic processes.

5. The method of paragraph 1, wherein the metabolic regulator is also combinations of 1 or more molecules, drugs, proteins, viral vectors, RNA-based systems, or CRISPR/CAS-based systems that specifically regulate metabolic processes.

6. The method of paragraph 1, wherein the source cell is directed to use pyruvate via glycolysis to generate GPA⁺ erythroid cells.

7. The method of paragraph 6, wherein pyruvate is blocked from entering mitochondria, thereby inhibiting tricarboxylic acid (TCA) cycle activity.

8. The method of paragraph 6, wherein the metabolic regulator is used to block:
(a) mitochondrial pyruvate carrier (MPC); or
(b) pyruvate dehydrogenase complex (PDH).

9. The method of paragraph 8, wherein UK5099 is used to inhibit MPC.

10. The method of paragraph 8, wherein 1-AA is used to inhibit PDH.

11. The method of paragraph 8, wherein the expression of MPC subunits (MPC1 and/or MPC2) is/are downregulated using shRNAs.

12. The method of paragraph 1, wherein the source cell is directed to promote OXPHOS or tricarboxylic acid (TCA) cycle activity to generate CD45⁺ hematopoietic cells.

13. The method of paragraph 12, wherein the metabolic regulator that promotes tricarboxylic acid (TCA) cycle activity is dimethyl α-ketoglutarate (DMK), alpha-ketoglutarate, or a related molecule.

14. The method of paragraph 12, wherein the source cell is directed to use pyruvate via oxidative phosphorylation (OXPHOS) and the source cell is differentiated into definitive CD45⁺ non-erythroid cells.

15. The method of paragraph 12, wherein the metabolic regulator promotes pyruvate use by the TCA cycle.

16. The method of paragraph 12, wherein DCA or a related molecule or an shRNA is used to inhibit pyruvate dehydrogenase kinases (PDKs) and thereby promote pyruvate use by OXPHOS.

17. The method of paragraph 12 comprising increasing pyruvate flux into mitochondria, which amplifies acetyl-CoA production and in turn promotes cholesterol metabolism and favors definitive hematopoietic output.

18. The method of paragraph 12, wherein the CD45⁺ non-erythroid cells are lymphoid cells.

19. The method of paragraph 18, wherein the lymphoid cells are T and/or B cells.

20. The method of paragraph 18, wherein the lymphoid cells are NK cells.

21. The method of paragraph 18, wherein the lymphoid cells are NKT cells.

22. The method of paragraph 18, wherein the lymphoid cells are lymphoid progenitors including common lymphoid progenitors (CLPs), pro-B cells, pre-B cells and thymocytes and NK cell progenitors and precursors.

23. A method of generating hematopoietic cells, comprising metabolically regulating a lipid biosynthesis pathway with an inhibitor of the lipid biosynthesis pathway.

24. The method of paragraph 23, wherein the inhibitor of the lipid biosynthesis pathway is CP-640186.

25. A method of generating hematopoietic cells comprising metabolically regulating a histone acetylation pathway with an inhibitor of the histone acetylation pathway.

26. The method of paragraph 25, wherein the inhibitor of the histone acetylation pathway is C646.

27. The method of paragraph 1, wherein the metabolic regulator includes an inhibitor or activator targeting glutaminolysis.

28. The method of paragraph 1, further comprising metabolically regulating alpha-ketoglutarate-dependent histone and DNA methylation with an inhibitor or activator targeting alpha-ketoglutarate-dependent histone and DNA methylation.

29. The method of paragraph 1, further comprising metabolically regulating a glutamine-dependent pathway with an inhibitor or activator targeting the glutamine-dependent pathway.

30. The method of paragraph 29, wherein the glutamine-dependent pathway is selected from the group consisting of nucleotide (purine and pyrimidine) biosynthesis, glutathione synthesis and non-essential amino acid synthesis.

31. A method of generating a hematopoietic cell, comprising:
providing a source cell; and
treating the source cell with cholesterol or a derivative of cholesterol,
wherein the source cell is differentiated into a CD45⁺ hematopoietic cell.

32. A method of generating a hematopoietic cell, comprising:
providing a source cell;
wherein the source cell is treated with an inhibitor or activator targeting the mevalonate pathway, and
wherein the source cell is differentiated into a GLY+ erythroid cell or a CD45⁺ hematopoietic cell, respectively.

33. A method of generating a hematopoietic cell comprising:
providing a source cell, and
metabolically regulating the source cell with fatty acids or a derivative of fatty acids.

34. A method of generating a hematopoietic cell comprising:
providing a source cell; and
metabolically regulating the source cell with a lipid or a derivative of a lipid.

35. A method of generating a hematopoietic cell, comprising:
providing a source cell; and
treating the source cell with an inhibitor or activator that targets a lipid biosynthesis pathway,
wherein the source cell is differentiated into a GPA⁺ erythroid cell or a CD45⁺ non-erythroid cell.

36. A method of providing hematopoietic cells to a subject with a malignancy or hematological disorder comprising:
(a) obtaining hematopoietic cells according to the method of paragraph 1; and
(b) transplanting the hematopoietic cells into the subject.

37. The method of paragraph 36, wherein the hematopoietic cells are modified to express chimeric antigen receptors (CAR).

38. The method of paragraph 36, wherein the malignancy is selected from the group consisting of leukemia (acute lymphocytic (ALL), chronic lymphocytic (CLL), acute myeloid (AML), chronic myeloid (CML)), myeloma, and lymphoma (Hodgkin's and non-Hodgkin's (NHL), Glioblastoma, glioma, pancreatic malignancies, or any other malignancy where a hematopoietic cell transplant could be used).

39. The method of paragraph 20, wherein the source cell is treated with a molecule that increases HMG-CoA reductase to increase cholesterol biosynthesis, thereby increasing output of NK cells.

40. The method of paragraph 39, wherein the molecule that increases HMG-CoA reductase is thyroid hormone.

41. The method of paragraph 40, wherein the thyroid hormone is added to the source cell during the HE stage to increase NK cell output.

1. A method of generating a hematopoietic cell, comprising:
providing a source cell;
treating the source cell with a metabolic regulator that directs the source cell to preferentially use glycolysis or oxidative phosphorylation; and
thereby obtaining a GPA+ erythroid cell or a CD45+ non-erythroid cell.

2-49. (canceled)
